SEQUENCE LISTING (98 sequences)

SEQ ID NO: 1 - protein, 9 aa, synthetic construct
  REGION 1..9: note = synthesized peptide
ATCYCRTGR

SEQ ID NO: 2 - protein, 9 aa, synthetic construct
  REGION 1..9: note = synthesized peptide
RGTRCYCTA

SEQ ID NO: 3 - protein, 11 aa, synthetic construct
  REGION 1..11: note = synthesized peptide
VCSCRLVFCRR

SEQ ID NO: 4 - protein, 11 aa, synthetic construct
  REGION 1..11: note = synthesized peptide
RRCFVLRCSCV

SEQ ID NO: 5 - protein, 9 aa, synthetic construct
  REGION 1..9: note = synthesized peptide
  MOD_RES 1: note = ACETYLATION
  REGION 1..7: note = MISC_FEATURE - D-amino acid
  MOD_RES 9: note = AMIDATION
  SITE 9: note = MISC_FEATURE - D-amino acid
ATCYCRTGR

SEQ ID NO: 6 - protein, 11 aa, synthetic construct
  REGION 1..11: note = synthesized peptide
  MOD_RES 1: note = ACETYLATION
  REGION 1..11: note = MISC_FEATURE - D-amino acid
  MOD_RES 11: note = AMIDATION
VCSCRLVFCRR

SEQ ID NO: 7 - DNA, 19 bp, synthetic construct
  misc_feature 1..19: note = primer
  source 1..19: mol_type = other DNA
gtgccagcmgccgcggtaa

SEQ ID NO: 8 - DNA, 20 bp, synthetic construct
  misc_feature 1..20: note = primer
  source 1..20: mol_type = other DNA
ggactachvgggtwtctaat

SEQ ID NO: 9 - protein, 32 aa, Homo sapiens
ATCYCRTGRCATRESLSGVCEISGRLYRLCCR

SEQ ID NO: 10 - protein, 29 aa, Homo sapiens
ATCYCRTGRCATRESLSGVCEISGRLYRL

SEQ ID NO: 11 - protein, 28 aa, Homo sapiens
CRTGRCATRESLSGVCEISGRLYRLCCR

SEQ ID NO: 12 - protein, 28 aa, Homo sapiens
ATCYCRTGRCATRESLSGVCEISGRLYR

SEQ ID NO: 13 - protein, 27 aa, Homo sapiens
ATCYCRTGRCATRESLSGVCEISGRLY

SEQ ID NO: 14 - protein, 26 aa, Homo sapiens
TGRCATRESLSGVCEISGRLYRLCCR

SEQ ID NO: 15 - protein, 26 aa, Homo sapiens
ATCYCRTGRCATRESLSGVCEISGRL

SEQ ID NO: 16 - protein, 25 aa, Homo sapiens
CRTGRCATRESLSGVCEISGRLYRL

SEQ ID NO: 17 - protein, 25 aa, Homo sapiens
ATCYCRTGRCATRESLSGVCEISGR

SEQ ID NO: 18 - protein, 24 aa, Homo sapiens
CRTGRCATRESLSGVCEISGRLYR

SEQ ID NO: 19 - protein, 23 aa, Homo sapiens
CATRESLSGVCEISGRLYRLCCR

SEQ ID NO: 20 - protein, 23 aa, Homo sapiens
CRTGRCATRESLSGVCEISGRLY

SEQ ID NO: 21 - protein, 22 aa, Homo sapiens
TGRCATRESLSGVCEISGRLYR

SEQ ID NO: 22 - protein, 22 aa, Homo sapiens
CRTGRCATRESLSGVCEISGRL

SEQ ID NO: 23 - protein, 21 aa, Homo sapiens
TGRCATRESLSGVCEISGRLY

SEQ ID NO: 24 - protein, 21 aa, Homo sapiens
CRTGRCATRESLSGVCEISGR

SEQ ID NO: 25 - protein, 19 aa, Homo sapiens
ESLSGVCEISGRLYRLCCR

SEQ ID NO: 26 - protein, 19 aa, Homo sapiens
CATRESLSGVCEISGRLYR

SEQ ID NO: 27 - protein, 19 aa, Homo sapiens
TGRCATRESLSGVCEISGR

SEQ ID NO: 28 - protein, 18 aa, Homo sapiens
CATRESLSGVCEISGRLY

SEQ ID NO: 29 - protein, 16 aa, Homo sapiens
SGVCEISGRLYRLCCR

SEQ ID NO: 30 - protein, 16 aa, Homo sapiens
ATCYCRTGRCATRESL

SEQ ID NO: 31 - protein, 15 aa, Homo sapiens
ESLSGVCEISGRLYR

SEQ ID NO: 32 - protein, 16 aa, Homo sapiens
CATRESLSGVCEISGR

SEQ ID NO: 33 - protein, 14 aa, Homo sapiens
ESLSGVCEISGRLY

SEQ ID NO: 34 - protein, 13 aa, Homo sapiens
ATCYCRTGRCATR

SEQ ID NO: 35 - protein, 13 aa, Homo sapiens
SGVCEISGRLYRL

SEQ ID NO: 36 - protein, 12 aa, Homo sapiens
CRTGRCATRESL

SEQ ID NO: 37 - protein, 12 aa, Homo sapiens
ESLSGVCEISGR

SEQ ID NO: 38 - protein, 11 aa, Homo sapiens
SGVCEISGRLY

SEQ ID NO: 39 - protein, 9 aa, Homo sapiens
CRTGRCATR

SEQ ID NO: 40 - protein, 10 aa, Homo sapiens
SGVCEISGRL

SEQ ID NO: 41 - protein, 7 aa, Homo sapiens
LYRLCCR

SEQ ID NO: 42 - protein, 6 aa, Homo sapiens
YRLCCR

SEQ ID NO: 43 - protein, 7 aa, Homo sapiens
TGRCATR

SEQ ID NO: 44 - protein, 6 aa, Homo sapiens
ATCYCR

SEQ ID NO: 45 - protein, 5 aa, Homo sapiens
RLCCR

SEQ ID NO: 46 - protein, 5 aa, Homo sapiens
CRTGR

SEQ ID NO: 47 - protein, 32 aa, Homo sapiens
AFTCHCRRSCYSTEYSYGTCTVMGINHRFCCL

SEQ ID NO: 48 - protein, 30 aa, Homo sapiens
TCHCRRSCYSTEYSYGTCTVMGINHRFCCL

SEQ ID NO: 49 - protein, 29 aa, Homo sapiens
AFTCHCRRSCYSTEYSYGTCTVMGINHRF

SEQ ID NO: 50 - protein, 28 aa, Homo sapiens
AFTCHCRRSCYSTEYSYGTCTVMGINHR

SEQ ID NO: 51 - protein, 27 aa, Homo sapiens
TCHCRRSCYSTEYSYGTCTVMGINHRF

SEQ ID NO: 52 - protein, 26 aa, Homo sapiens
TCHCRRSCYSTEYSYGTCTVMGINHR

SEQ ID NO: 53 - protein, 25 aa, Homo sapiens
RSCYSTEYSYGTCTVMGINHRFCCL

SEQ ID NO: 54 - protein, 24 aa, Homo sapiens
SCYSTEYSYGTCTVMGINHRFCCL

SEQ ID NO: 55 - protein, 23 aa, Homo sapiens
AFTCHCRRSCYSTEYSYGTCTVM

SEQ ID NO: 56 - protein, 22 aa, Homo sapiens
RSCYSTEYSYGTCTVMGINHRF

SEQ ID NO: 57 - protein, 21 aa, Homo sapiens
TCHCRRSCYSTEYSYGTCTVM

SEQ ID NO: 58 - protein, 21 aa, Homo sapiens
RSCYSTEYSYGTCTVMGINHR

SEQ ID NO: 59 - protein, 21 aa, Homo sapiens
SCYSTEYSYGTCTVMGINHRF

SEQ ID NO: 60 - protein, 21 aa, Homo sapiens
STEYSYGTCTVMGINHRFCCL

SEQ ID NO: 61 - protein, 20 aa, Homo sapiens
SCYSTEYSYGTCTVMGINHR

SEQ ID NO: 62 - protein, 17 aa, Homo sapiens
AFTCHCRRSCYSTEYSY

SEQ ID NO: 63 - protein, 18 aa, Homo sapiens
STEYSYGTCTVMGINHRF

SEQ ID NO: 64 - protein, 17 aa, Homo sapiens
STEYSYGTCTVMGINHR

SEQ ID NO: 65 - protein, 17 aa, Homo sapiens
SYGTCTVMGINHRFCCL

SEQ ID NO: 66 - protein, 15 aa, Homo sapiens
TCHCRRSCYSTEYSY

SEQ ID NO: 67 - protein, 15 aa, Homo sapiens
AFTCHCRRSCYSTEY

SEQ ID NO: 68 - protein, 15 aa, Homo sapiens
GTCTVMGINHRFCCL

SEQ ID NO: 69 - protein, 13 aa, Homo sapiens
TCHCRRSCYSTEY

SEQ ID NO: 70 - protein, 14 aa, Homo sapiens
SYGTCTVMGINHRF

SEQ ID NO: 71 - protein, 13 aa, Homo sapiens
SYGTCTVMGINHR

SEQ ID NO: 72 - protein, 11 aa, Homo sapiens
AFTCHCRRSCY

SEQ ID NO: 73 - protein, 12 aa, Homo sapiens
STEYSYGTCTVM

SEQ ID NO: 74 - protein, 12 aa, Homo sapiens
GTCTVMGINHRF

SEQ ID NO: 75 - protein, 10 aa, Homo sapiens
RSCYSTEYSY

SEQ ID NO: 76 - protein, 11 aa, Homo sapiens
GTCTVMGINHR

SEQ ID NO: 77 - protein, 9 aa, Homo sapiens
TCHCRRSCY

SEQ ID NO: 78 - protein, 9 aa, Homo sapiens
SCYSTEYSY

SEQ ID NO: 79 - protein, 9 aa, Homo sapiens
GINHRFCCL

SEQ ID NO: 80 - protein, 8 aa, Homo sapiens
RSCYSTEY

SEQ ID NO: 81 - protein, 8 aa, Homo sapiens
AFTCHCRR

SEQ ID NO: 82 - protein, 8 aa, Homo sapiens
SYGTCTVM

SEQ ID NO: 83 - protein, 7 aa, Homo sapiens
SCYSTEY

SEQ ID NO: 84 - protein, 7 aa, Homo sapiens
AFTCHCR

SEQ ID NO: 85 - protein, 6 aa, Homo sapiens
TCHCRR

SEQ ID NO: 86 - protein, 6 aa, Homo sapiens
STEYSY

SEQ ID NO: 87 - protein, 6 aa, Homo sapiens
GINHRF

SEQ ID NO: 88 - protein, 5 aa, Homo sapiens
TCHCR

SEQ ID NO: 89 - protein, 6 aa, Homo sapiens
GTCTVM

SEQ ID NO: 90 - protein, 4 aa, Homo sapiens
RSCY

SEQ ID NO: 91 - protein, 9 aa, synthetic construct
  REGION 1..9: note = Synthesized peptide
  SITE 3: note = MISC_FEATURE - X is (2S)-2-aminobutanoic acid (Abu)
ATXYCRTGR

SEQ ID NO: 92 - protein, 9 aa, synthetic construct
  REGION 1..9: note = Synthesized peptide
  SITE 5: note = MISC_FEATURE - X is (2S)-2-aminobutanoic acid (Abu)
ATCYXRTGR

SEQ ID NO: 93 - protein, 9 aa, synthetic construct
  REGION 1..9: note = Synthesized peptide
  SITE 3: note = MISC_FEATURE - X is (2S)-2-aminobutanoic acid (Abu)
  SITE 5: note = MISC_FEATURE - X is (2S)-2-aminobutanoic acid (Abu)
ATXYXRTGR

SEQ ID NO: 94 - protein, 9 aa, synthetic construct
  REGION 1..9: note = Synthesized peptide
  SITE 6: note = MISC_FEATURE - X is citrullin (Cit)
ATCYCXTGR

SEQ ID NO: 95 - protein, 9 aa, synthetic construct
  REGION 1..9: note = Synthesized peptide
  SITE 9: note = MISC_FEATURE - X is citrullin (Cit)
ATCYCRTGX

SEQ ID NO: 96 - protein, 9 aa, synthetic construct
  REGION 1..9: note = Synthesized peptide
  SITE 6: note = MISC_FEATURE - X is citrullin (Cit)
  SITE 9: note = MISC_FEATURE - X is citrullin (Cit)
ATCYCXTGX

SEQ ID NO: 97 - protein, 9 aa, synthetic construct
  REGION 1..9: note = Synthesized peptide
RGTRCYCTA

SEQ ID NO: 98 - protein, 9 aa, synthetic construct
  REGION 1..9: note = Synthesized peptide
CTRATYCRG

(19) United States
(12) Patent Application Publication
(10) Pub. No.: US 2025/0368685 A1
(43) Pub. Date: Dec. 4, 2025

(54) DEFENSIN FRAGMENTS FOR USE IN THERAPY OR PROPHYLAXIS

(71) Applicant: Aesculus Bio ApS, Copenhagen N (DK)

(72) Inventors: Jan Wehkamp, Reutlingen (DE); Dirk Ehmann, Tübingen (DE)

(73) Assignee: Aesculus Bio ApS, Copenhagen N (DK)

(21) Appl. No.: 19/232,282

(22) Filed: Jun. 9, 2025

Related U.S. Application Data
(63) Continuation of application No. 17/420,155, filed on Jul. 1, 2021, now Pat. No. 12,358,949.

(30) Foreign Application Priority Data
Jan. 7, 2019 (DE) 102019100230.3
Apr. 2, 2019 (DE) 102019108626.4

(51) Int. Cl. C07K 7/08; A61K 38/00

(52) U.S. Cl. CPC C07K 7/08; A61K 38/00

(57) ABSTRACT

The present invention relates to new peptides derived from HD-5 or HNP-4 having antimicrobial activity for use in modulating the microbiome of intestines, the lungs, the skin, the mouth, the eye, the ear, the vagina or other bodily surfaces and/or for use as an antimicrobial agent in a human or other mammals, as well as to medicaments containing these peptides.

### CROSS REFERENCE TO RELATED APPLICATIONS

This application is a continuation of U.S. patent application Ser. No. 17/420,155 filed Jul. 1, 2021, which is a U.S. national stage under 35 U.S.C. § 371 of International Application No: PCT/EP2020/050186 filed Jan. 7, 2020, and which claims priority to German Application No: 10 2019 108 626.4 filed Apr. 2, 2019 and German Application No: 10 2019 100 230.3 filed Jan. 7, 2019, the entire contents of each of which are incorporated by reference.

### FIELD

The present invention relates to specific peptide sequences derived from alpha defensins and their use in medical therapy and/or prophylaxis.

Reference to Sequence Listing Submitted Electronically The instant application contains a Sequence Listing which has been submitted electronically in xml format and is hereby incorporated by reference in its entirety. Said .xml copy, created Jun. 6, 2025, is named “AEBI0001NA Sequence Listing.xml” and is 97,634 bytes in size.

### BACKGROUND

Anaerobic and aerobic microorganisms, especially bacteria and yeasts, i.e. unicellular microorganisms that live with oxygen, without oxygen or are oxygen-tolerant, can cause various clinical pictures, for example wound infections and abscesses, sepsis, infections, especially in the abdominal cavity, in the urogenital tract, on the skin or in the mouth, eyes, ears, and jaw region. Thus, these pathogenic species are often already found in the region of the skin and oral cavity, especially in the inflamed skin/eczema, the periodontium, the eyes and ears and in the region of the stomach in the mucosal folds of the stomach, and in the duodenum, which can then cause local, but also in some circumstances systemic acute and chronic inflammations. Even in the rather thinly colonized small intestine, a number of facultative anaerobes can cause pathological changes of the highly sensitive mucosa of the small intestine; in the rectum, the principal site of the bacterial flora, admittedly aerobic bacteria predominate, but here too, anaerobicepresentatives are also capable of causing serious inflammatory reactions of the mucosa of the colon. Candida ssp. are also found in the stool of many individuals and are potentially pathogenic.

At present, in particular such diseases are treated with antibiotics, which mainly attack and destroy the cell walls of the bacteria. A big problem that arises when these inflammatory diseases are treated with antibiotics is the development of resistance to the antibiotics used, which in recent times has progressed even further. This enables the pathogenic bacteria/microorganisms to weaken or completely neutralize the action of antibiotic substances. If a microorganism then proves to be resistant to the common antibiotics, diseases can become life-threatening. The reason why in the past the number of multi resistant bacterial strains has increased considerably is that, owing to their rapid growth and their short culture period, the bacteria are continually able to develop new strategies for neutralizing the antibiotics. Therefore at present, in addition to antibiotics, for example also natural, especially plant, and synthetic oils and emulsions are used.

In recent years, antimicrobial peptides, which are part of the natural immune system and are vitally important for epithelial defense against infection by microorganisms, have gained research and therapeutic appliance interests.

In a healthy person the skin and mucosa form a physical barrier to infection by microorganisms. The physical barrier is made up of the stratum corneum in healthy skin and, in the mucosa, of the mucous layer in which desquamation and mucous secretion cause a constant renewal of the surfaces, simultaneously with continuous elimination of microorganisms that are adhering to the surfaces. In interaction with the lipids that are also present in the skin, this physical barrier prevents microorganisms from penetrating into the living epidermis.

Leaving aside this physical barrier, however, further factors are also necessary in order for the healthy skin and mucosa to defend against infection; among these factors are endogenous antimicrobial peptides (AMPs). Lysozyme, for example, is an antimicrobial peptide that is present in nasal secretions and can in particular kill Gram-positive bacteria. Also known as antimicrobial peptides in the intestinal mucosa are defensins, whose presence appears to be necessary especially given that the intestinal epithelia is exposed to very large quantities of bacteria. In addition to having a mucous layer that is difficult for microorganisms to penetrate, the intestinal mucosa contains Paneth cells that secrete human defensin-5—an alpha defensin—that among other functions, protect the stems cells that are important for continuous renewal of the intestinal mucosa. In humans, only alpha- and beta-defensins are expressed. While alpha-defensins are expressed primarily in neutrophils as well as in NK cells and certain T-lymphocyte subsets, human defensin 5 and defensin 6 are expressed exclusively in Paneth cells of the small intestine, where they contribute in regulating and maintaining microbial balance in the intestinal lumen. On the other hand, beta-defensins are most widely distributed, being secreted by leukocytes and epithelial cells of many kinds. Further known AMPs are a peptide known as psoriasin, as well as RNas-7, which represents an effective endogenous broad-spectrum antibiotic in humans.

In addition to the known endogenous antimicrobial peptides, numerous antibiotics are also known in the existing art; these include both substances of biological origin and synthetically manufactured substances, which are therefore either (as in the original sense) naturally formed low-molecular-weight metabolic products of fungi or bacteria, or chemically synthesized therapeutic agents.

Especially in light of the fact that the development of resistance to natural and synthetic antibiotics is making microbial infectious diseases increasingly difficult to treat, a need also frequently arises for novel antimicrobial active agents that are notable for few side effects and for simple manufacture and handling.

The gastrointestinal microenvironment is comprised of a single cell layer epithelia, a mucus layer, a local immune system, and the microbiome, and together these four components play a crucial role in maintaining homeostasis during times of health. The human colon harbors a highly dense microbial community of 10¹¹-10¹² cells per gram of gut content, and human health is closely linked to the diverse set of microorganisms in the intestine collectively known as the gut microbiota. While on one hand, their abundance and prevalence are associated with disease—as it can be the case with Faecalibacterium prausnitzii in, e.g., inflammatory bowel disease (IBD) and infectious colitis, it has been shown, on the other hand, that mucosal species such as Bacteroides fragilis, and Lactobacillus reuteri can protect against colitis.

As a consequence, when applying antibiotics in case of an infection, the microbiome composition in human colon gets majorly affected, and the microbe balance disturbed. Defensins are small cationic molecules, characterized by three conserved disulphide bonds and represent a main group of AMPs. To date, six alpha-defensins have been identified in humans, namely the four Human Neutrophil Peptides (HNP) 1, 2, 3 and 4, and the two Human Defensin (HD) 5 and 6. While the HNPs form part of the armory of neutrophils, where they participate in systemic innate immunity, the HDs are expressed in intestinal Paneth cells. As mentioned above, in the small intestine, Paneth cells play a key role in balancing the microbiota composition and in protecting the host from invading pathogens by secretion of a variety of AMPs but most abundantly the two α-defensin 5 (HD 5) and -6 (HD 6).

While HNP-1, HNP-2 and HNP-3 only differs in a single amino acid, HNP-4 varies in its sequence, has one additional positive charge and exhibits improved bactericidal activity compared to HNP-1-3^{1,2}. The activity of full length antimicrobial peptides are influenced by environmental conditions including salt concentration, pH or redox potential³⁻⁶. Based on its strong antimicrobial activity we used HNP-4 as a precursor for a new therapeutic agent with antimicrobial abilities. While large-scale expression of accurately folded defensins is a major issue, we focused on small fragments of HNP-4. We used a natural occurring protease to digest the full length peptide and subsequently identified the generated fragments. We tested these fragments for their antibacterial and antifungal potential and analyzed their cytotoxic and hemolytic abilities.

The antimicrobial activity of alpha-defensins has been intensively studied in the past, and it has been acknowledged that alterations in their specific sequences can contribute to major changes of their activity and may even lead to a complete loss of antimicrobial activity.

### SUMMARY

The problem to be solved by the present invention is therefore to provide a new or alternative preventive and/or therapeutic approach, by means of which infectious diseases but also other diseases associated with dysbiotic conditions, e.g. metabolic diseases, lung diseases, urogenital diseases, diseases of the mouth, eyes and ears and skin diseases, can be prevented and/or effectively treated.

According to the invention, this and other problems are solved by the provision of peptides having antimicrobial activity and having an amino acid sequence derived from an alpha-defensin fragment, which peptide consists of between 6 and 27, in particular the shorter peptide fragments, which can be synthesized as linear peptides, for example those having 7, 9, 11, or 13, successive amino acids.

Common for the peptides is that they are fragments of naturally occurring alpha defensins, HD-5 and HNP-4 and can be produced by reducing the naturally occurring peptides and subjecting them to cleavage using proteases activity. Surprisingly, a related peptide, HD-6 which includes a number of predicted cleavage sites, could not be cleaved under the same conditions.

These short fragments of naturally occurring alpha-defensins have the advantage that they can be synthesized chemically as small linear peptide thus reducing the cost significantly compared to the manufacture of full length peptides. Furthermore, several of the peptides have retained the antibiotic effects of the naturally occurring full length defensins, while being non-toxic at efficacious concentrations.

These peptides can be used for modulating the microbiome of intestines and/or as an antimicrobial agent without inducing major shifts/disturbing the balance of the healthy microbiome.

Within the present invention, peptide sequences of alpha-defensins have been identified which, in antimicrobial tests, showed to have an increased antimicrobial effect on certain (in particular: pathogenic) bacteria as compared to the full length peptide on one hand, while on the other hand there was no effect of the newly identified peptides on the microbial diversity.

These results allow the appliance of the peptides according to the invention not only for treating microbial infections and bacterially caused diseases, even those diseases that are caused by antibiotic-resistant bacteria, but also for preventing bacterial infections and for modulating the gut microbiome and potentially the microbiome of other epithelial surfaces e.g. lung, skin, genitourinary tract, mouth, eyes, ears etc.

Accordingly, within the present invention, and as generally understood in the field, “modulating the microbiome” means the beneficial influence of the peptides on the microorganisms present in the gut and epithelial surfaces. As mentioned above, gut microbes are key to many aspects of human health including immune, metabolic and neuro behavioral traits. With the peptides for use according to the invention, the bacterial diversity of the gut microbiome and potentially the bacterial diversity of other epithelial surfaces can be supported and promoted.

With “gut microbiome” the gut of a mammal, in particular of a human, is meant. Accordingly, a preferred embodiment of the invention is directed to the peptide for use in modulating the gut microbiome of humans.

According to an embodiment of the peptide of the invention the alpha-defensin fragment is a fragment of HD-5 or HNP4.

HD-5, as mentioned at the outset, is expressed in Paneth cells of the small intestine. Including a signal peptide and a prodomain, HD 5 comprises 94 amino acids, with the mature peptide comprising amino acid numbers 63 to 94.

HNP4, also as mentioned at the outset, expressed in the granules of neutrophils. Including the signal peptide and a prodomain, HNP4 comprises 97 amino acids, with the mature HNP4 peptide comprising amino acid numbers 64 to 96.

According to a preferred embodiment of the invention, the peptide for use according to the invention consists of between 6 and 27 successive amino acids derived from HD-5 and consists of
the sequence HD-5₁₋₉ ATCYCRTGR (SEQ ID No. 1) or
the reverse sequence of SEQ ID No. 1, RGTRCYCTA (SQ ID No. 2),
modified HD-5₁₋₉: Ac-atcycrtGr-NH₂ (SEQ ID No. 5),
HD-5₁₋₁₃, ATCYCRTGRCATR (SEQ ID No. 34),
HD-5₁₋₂₈, ATCYCRTGRCATRESLSGVCEISGRLYR (SEQ ID No. 12),
HD-5₇₋₃₂, TGRCATRESLSGVCEISGRLYRLCCR (SEQ ID No. 14
HD-5₁₀₋₃₂, CATRESLSGVCEISGRLYRLCCR (SEQ ID No. 19,
HD-5₁₄₋₃₂, ESLSGVCEISGRLYRLCCR (SEQ ID No. 25
HD-5₁₀₋₂₇ CATRESLSGVCEISGRLY (SEQ ID No. 28) or
HD-5₂₆₋₃₂ LYRLCCR (SEQ ID No. 41) of the attached sequence listing.

In general for amino acid sequences, capital letters designate L-amino acids and small letters designate D-amino acids.

In a preferred embodiment the peptide consists of the sequence of
ATCYCRTGR (SEQ ID No. 1),
RGTRCYCTA (SEQ ID No. 2),
Ac-atcycrtGr-NH₂ (SEQ ID No. 5),
LYRLCCR (SEQ ID No. 41),
ATCYCRTGRCATR (SEQ ID No. 34),
ATCYCRTGRCATRESLSGVCEISGRLYR (SEQ ID No. 12), or
TGRCATRESLSGVCEISGRLYRLCCR (SEQ ID No. 14).

More preferably the peptide consists of the sequence of
ATCYCRTGR (SEQ ID No. 1),
RGTRCYCTA (SEQ ID No. 2),
Ac-atcycrtGr-NH₂ (SEQ ID No. 5), or
LYRLCCR (SEQ ID No. 41).

Preferred peptide include those based on HD-5₁₋₉:

(SEQ ID No. 1)
ATCYCRTGR,
(SEQ ID No. 2)
RGTRCYCTA, or
(SEQ ID NO. 5)
Ac-atcycrtGr-NH₂.

According to a preferred embodiment of the invention, the peptide for use according to the invention consists of either 7, 9, 11, or 13 successive amino acids.

According to a preferred embodiment of the invention, the peptide for use according to the invention consists of 9 successive amino acids derived from HD-5, and preferably consists of the sequence ATCYCRTGR (SEQ ID No. 1) or the reverse sequence of SEQ ID No. 1, RGTRCYCTA (SQ ID No. 2) of the attached sequence listing.

According to another preferred embodiment of the invention, the peptide for use according to the invention consists of 11 successive amino acids derived from HNP4, and preferably consists of the sequence VCSCRLVFCRR (SEQ ID No. 3), of the reverse sequence of SEQ ID No. 3, RRCFVLRCSCV (SEQ ID No. 4), or of the modified HNP-4₁₋₁₁: Ac-vcscrlvfcrr-NH₂ (SEQ ID NO. 6).

In one embodiment, the invention relates to a method of manufacturing the peptides of the invention comprising subjecting reduced HD5 or HNP-4 to protease activity, e.g. trypsin or chymotrypsin, followed by purification.

The peptides as herein disclosed and described, being derived from HD-5 or HNP4, have been shown to exhibit excellent antimicrobial activities against pathogenic bacteria, while at the same time not notably influencing the commensal microbiota, e.g. the gut microbiota.

According to a preferred embodiment, the peptide for use according to the invention comprises L- and/or D-amino acids.

Presently and as generally understood, an “L-amino acid” refers to a stereoisomer of a particular amino acid whose amino group is on the left side in its Fisher projection while D-amino acid refers to the other stereoisomer of the amino acid whose amino group is on the right side in its Fisher projection. In sequences herein, L-amino acids are shown in capital and D-amino acids in small letters.

While most naturally occurring peptides are composed of amino acids in the L-configuration, D-amino acids have shown strong resistance to proteolytic degradation.

Thus, according to a preferred embodiment, the peptides according to the invention consist of D-amino acids.

According to another embodiment, the peptides according to the invention consist of L-amino acids.

According to another embodiment, the peptides according to the invention consist of a mixture of D- and L-amino acids, preferably alternating D- and L-amino acids, or comprises preferably one L-amino acid, with the remaining amino acids being D-amino acids.

According to a preferred embodiment, the peptide for use according to the invention comprises N- and/or C-terminal modifications.

With N- and/or C-terminal modifications it is possible to influence/enhance, e.g., the stability or the half-life of the peptide according to the invention, in particular in environments that promote degradation and/or modification of the free N-/C-terminal ends of peptides, e.g. due to proteases present in those environments.

Presently, and as generally understood, the C-terminus (also known as the carboxyl-terminus, carboxy-terminus, C-terminal tail, C-terminal end, or COOH-terminus) is the end of an amino acid chain (protein or polypeptide), terminated by a free carboxyl group (—COOH), and the N-terminus (also known as the amino-terminus, NH₂-terminus, N-terminal end or amine-terminus) is the start of a protein or polypeptide referring to the free amine group (—NH₂) located at the end of a polypeptide. The convention for writing peptide sequences is to put the C-terminal end on the right and write the sequence from N- to C-terminus.

According to a preferred embodiment, in the peptide for use according to the invention, the C-terminal modification is selected from one of the group consisting of: -Amide, -Acid, -N-alkyl-Amide, -Aldehyde, -Ester, -p-Nitroanilide, and -7-Amino-4-Methylcoumarin.

With these modifications, the C-terminal end of the peptide can be protected without influencing the antimicrobial activity of the peptide to a major extent.

According to a preferred embodiment, in the peptide for use according to the invention, the N-terminal modification is selected from one of the group consisting of Acetyl-, Formyl-, Pyroglutamyl-, Fatty acids-, urea-, Carbamate-, and alkylamine-.

It is to be understood, that either both ends, i.e. the N-terminus and the C-terminus can be modified with any of the above described modifications, or only one of the ends, i.e. either the N- or the C-terminus.

According to a preferred embodiment, in the peptide for use according to the invention, the N-terminus, carries an acetyl-(ac) modification, and no modification at the C-terminus.

According to a preferred embodiment, the peptide for use according to the invention, the peptide consists of (or comprises) D-amino acids, and carries, at the N-terminus, an acetyl-(ac) modification, and no modification at the C-terminus.

With an N-terminal acetylation, the charge from the amino terminus of a peptide is removed; also, with an acetyl modification, a peptide is meant to imitate its natural structure in a protein. In addition, this modification stabilizes the resulting peptide towards enzymatic degradation resulting from exopeptidases.

According to a preferred embodiment, the peptide according to the invention carries a N-terminal acetyl-modification and a C-terminal amide-modification.

With a C-terminal amide modification, the peptide is meant to imitate its natural structure in a protein. In addition, this modification avoids the introduction of additional charges in the peptide molecule.

According to a preferred embodiment, the peptide according to the invention comprises 9 amino acids, wherein 8 amino acids are D-amino acids, and 1 amino acid is a L-amino acid, and further preferably, this peptide comprises an N-terminal acetyl-modification and a C-terminal amide-modification.

According to a preferred embodiment, the peptide according to the invention comprises 11 amino acids, wherein all amino acids are D-amino acids, and further preferably this peptide comprises an N-terminal acetyl-modification and a C-terminal amide-modification.

According to another preferred embodiment, the peptide according to the invention is a chemically synthesized peptide or a biologically expressed peptide.

A wide variety of methods for chemically synthesizing peptides are known in the art; while the chemical synthesis of peptides can be carried out using classical solution-phase techniques, these have been replaced in most research and development settings by solid-phase methods. An overview of peptide synthesis can be found, e.g. in Stawikowski et al., (“Introduction to peptide synthesis”, Cur. Prot. Prot. Sci., (2012), suppl. 69, 18.1.1-18.1.13)⁷.

According to an embodiment of the peptide for use of the invention, the peptide is in an oxidized or reduced state.

In this connection, “oxidized” refers to the state of the peptide where disulfide bridges, which occur in peptides having amino acid residues such as cysteine, methionine, tryptophan, histidine and tyrosine. The “reduced” state or from designates the form of the peptide not having formed disulfide bonds.

“Biologically expressed” peptide, within the present invention, shall encompass the expression of the peptide(s) for use according to the invention by a genetically engineered host cell that has been modified to express said peptide(s).

As used herein, the term “host cell” is presently defined as a cell which has been transformed or transfected, or is capable of transformation or transfection by an exogenous polynucleotide sequence encoding the peptide for use according to the invention.

A variety of host-expression vector systems may be utilized to express the gene coding a peptide for use according to the invention. Such host-expression systems represent vehicles by which the coding sequences of interest may be produced and subsequently purified, but also represent cells which, when transformed or transfected with the appropriate nucleotide coding sequences, exhibit the peptide for use gene product of the invention in situ.

For recombinant production, host cells can be genetically engineered to incorporate expression systems or portions thereof or polynucleotides encoding the peptides for use of the invention. Introduction of a polynucleotide into the host cell can be effected by methods described in many standard laboratory manuals, such as Davis et al., Basic Methods in Molecular Biology, (2012)⁸, and Sambrook et al., 1989⁹.

Thus, the polynucleotide encoding the peptide according to the invention, may, e.g., be comprised in a vector which is to be stably transformed/transfected into host cells. In the vector, the polynucleotide encoding the peptide(s) of the invention is under control of an, e.g., inducible promoter, so that the expression of the gene/polynucleotide can be specifically targeted, and, if desired, the gene may be overexpressed in that way.

A great variety of expression systems can be used to produce the polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above

According to a preferred embodiment, the peptide for use according to the invention is selected from at least one of the followings:

HD-5₁₋₉:
(SEQ ID No. 1)
ATCYCRTGR
HD-5₁₋₉ᵣₑᵥ:
(SEQ ID No. 2)
RGTRCYCTA
HD-5_{1-9mod}:
(SEQ ID No. 5)
Ac-atcycrtGr-NH₂
HNP-4₁₋₁₁:
(SEQ ID No. 3)
VCSCRLVFCRR
HNP-4₁₋₁₁ᵣₑᵥ:
(SEQ ID No. 4)
RRCFVLRCSCV
HNP-4_{1-11mod}:
(SEQ ID NO. 6)
Ac-vcscrlvfcrr-NH₂

According to another aspect of the invention, the use of the peptide in modulating the microbiome consists of the use in the treatment and/or prophylaxis of intestinal, lung, urogenital, mouth, eye, ear or skin or other conditions or diseases associated with a dysbiotic condition.

As mentioned above, a healthy gut microbiome, containing diverse bacterial microorganisms, is mandatory not only for an intact gut, but for overall health in mammals, in particular humans. A lower bacterial diversity has been reproducibly observed in people with diseases such as, inter alia, inflammatory bowel disease, coeliac disease, but also metabolic diseases like obesity and type 2 diabetes and the efficacy of e.g. check point inhibitor treatment of cancer is also highly influenced by the microbiome and even CNS diseases like schizophrenia has been reported to be influenced by the microbiome. The association between reduced diversity and disease indicates that a species-rich gut ecosystem is more robust against environmental influences, as functionally related microbes in an intact ecosystem can compensate for the function of other missing species.

Apart from genetically influenced intestinal diseases, also specific food and dietary patterns as well as medications can influence the abundance of different types of bacteria in the gut. While often associated with a change of the nutrition or diet positive effects on the gut microbiome can be observed, and also by using pre- and probiotic foods, the peptide according to the invention, due to its natural origin, provides for a broader, more convenient and highly efficient tool. Also with the peptide according to the invention, a treatment of subjects is possible that are otherwise highly sensitive towards nutritional changes and influences.

With the peptide for use according to the invention, intestinal diseases but also diseases of the lungs, the skin and the brain can be efficiently prevented and/or treated, by positively influencing the natural microbiome of the gut.

Thus, in a preferred embodiment, the peptide according to the invention is used in preventing/treating a disease that is selected from inflammatory bowel disease, in particular Crohn's disease, ulcerative colitis, coeliac disease, necrotizing enterocolitis, irritable bowel syndrome, tourist diarrhea, gastro-intestinal cancers and intestinal graft versus host disease, and from metabolic diseases, preferably diabetes and pre-diabetes, obesity, NAFLD, NASH, dyslipidemia, and from diseases of the lung, preferably asthma and COPD, and from diseases of the brain, preferably schizophrenia, Parkinsonism, bipolar disorder, autism and depression.

With the peptide according to the invention, diseases associated with the simple microbiomes of the skin, mouth, eye, ear, vagina or circulation can be efficiently prevented and/or treated.

Thus, in a preferred embodiment, the peptide according to the invention is used in preventing/treating a disease that is selected from sepsis, atopic dermatitis, rosacea, seborrheic dermatitis, eczema, carbuncles, staph infection, candidiasis, cellulitis, impetigo, acne, pilonidal cyst, Athletes food, ringworm, molluscum, cutaneous lymphoma, periodontitis, caries, Dry eye, Sjögrens Disease, conjunctivitis, blepharitis, hordeola, chalazia, periorbital cellulitis, dacryocystitis, endolphalmitis, uveitis, iritis, mastoiditis, vestibular neuronitis, bullous myringitis, granular myringitis, otitis externa, otitis media, bacterial vaginosis, Trichomonas vaginitis, candida, non-infectious vaginitis, inflammatory vaginitis.

According to another aspect, the peptide as described herein can also be used as an antimicrobial agent against multi drug resistant bacteria induced infections. Within the present invention it has been found that the peptide according to the invention can not only be used to positively influence the natural gut microbiome, but also as a tool for specifically targeting multi drug resistant bacteria, thus, being an efficient tool for treating/preventing infections caused by multi-drug resistant bacteria. According to another object, the present invention also relates to a medicament comprising the peptide according to the invention and a pharmaceutically acceptable carrier.

Presently, and as generally understood in the field, a “pharmaceutically acceptable carrier” is understood to mean any excipient, additive, or vehicle that is typically used in the field of the treatment of the mentioned diseases and which simplifies or enables the administration of the product according to the invention to a living being, and/or improves its stability and/or activity. The pharmaceutical composition can also incorporate binding agents, diluting agents or lubricants. The selection of a pharmaceutical carrier or other additives can be made on the basis of the intended administration route and standard pharmaceutical practice. As pharmaceutical acceptable carrier use can be made of solvents, extenders, or other liquid binding media such as dispersing or suspending agents, surfactant, isotonic agents, spreaders or emulsifiers, preservatives, encapsulating agents, solid binding media, depending upon what is best suited for the respective dose regime and is likewise compatible with the compound according to the invention. An overview of such additional ingredients can be found in, for example, Rowe (Ed.) et al.: Handbook of Pharmaceutical Excipients, 7th edition, 2012, Pharmaceutical Press¹⁰.

A peptide of the present invention can be used for treatment of skin-conditions when formulated for topical administration. Methods for topical administration are known in the art.

When formulated for topical administration, the composition of the present invention may contain ingredients typical in topical pharmaceutical or cosmetic compositions, such as a carrier, vehicle or medium. Specifically, the carrier, vehicle, or medium is compatible with the tissues to which it will be applied, such as the skin, hair, nail, vagina, urethra, ear, oral cavity, nasal passage, respiratory system, opthalmic region, and/or mucosa. The compositions and components of the invention are suitable for contacting infected tissues or for use in patients in general without undue toxicity, incompatibility, instability, allergic response, and the like. As appropriate, compositions of the invention may comprise any ingredient conventionally used in the fields under consideration.

In terms of their form, compositions of this invention may include solutions, emulsions (including microemulsions), suspensions, creams, lotions, gels, powders, or other typical solid or liquid compositions used for application to skin and other tissues where the compositions may be used. Such compositions may contain: additional antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals. In certain embodiments, the composition of the invention is formulated with the above ingredients so as to be stable for a long period of time, as may be beneficial where continual or long-term treatment is intended.

The compositions of the invention may be in the form of controlled-release or sustained-release compositions, wherein the antimicrobial peptide along with additional active agents are encapsulated or otherwise contained within a material, such that they are released onto the skin or affected area in a controlled manner over time. The compositions of the invention may be contained within or on matrixes, liposomes, vesicles, microcapsules, microspheres and the like, or within or on a solid particulate material.

Administration of the composition of the invention may be to any affected or susceptible region, for example, to the legs, shoulders, back (including lower back), axilla, palms, feet, neck, groin, dorsa or the hands or feet, elbows, upper arms, knees, upper legs, buttocks, torso, pelvis, or any other part of the body for which treatment or prevention of infection may be desired. Such treatment is also contemplated for treating and/or dressing wounds, such as cuts, scrapes, and burns to the skin, so as to treat or prevent infection of the wounded area.

The compositions of the invention are suitable for use in physiologic environments with a pH ranging from about 4.5 to about 6.3, and thus, the compositions may be formulated at a similar or equivalent pH. The compositions according to this invention may be stored either at room temperature or under refrigerated conditions. The composition of the invention contains an amount of antimicrobial peptide effective for antimicrobial action. Generally, the composition contains from about 0.01% (wt./vol.) to about 20% antimicrobial peptide. In certain embodiments, the composition contains from about 0.5% to about 10% antimicrobial peptide, such as about 0.5%, about 1%, about 5%, or about 10% antimicrobial peptide.

The features, characteristics and advantages of the peptide according to the invention apply likewise to the medicament according to the invention. Accordingly, the medicament containing the peptide according to the invention can also be used for treating and/or preventing a disease that is selected from inflammatory bowel disease, in particular Crohn's disease, ulcerative colitis, coeliac disease, necrotizing enterocolitis, irritable bowel syndrome, tourist diarrhea, gastro-intestinal cancers and intestinal graft versus host disease, but also metabolic diseases, preferably diabetes and pre-diabetes, obesity, NAFLD, NASH, dyslipidemia, and diseases of the lung, preferably asthma and COPD; and diseases of the skin such as atopic dermatitis, rosacea, seborrheic dermatitis, eczema, carbuncles, staph infection, candidiasis, cellulitis, impetigo, acne, pilonidal cyst, Athletes food, ringworm, molluscum, cutaneous lymphoma; diseases of the mouth such as periodontitis and caries; diseases of the eye such as Dry eye, Sjögrens Disease, conjunctivitis, blepharitis, hordeola, chalazia, periorbital cellulitis, dacryocystitis, endolphalmitis, uveitis, iritis; diseases of the ear such as mastoiditis, vestibular neuronitis, bullous myringitis, granular myringitis, otitis externa, otitis media; diseases of the vagina such as bacterial vaginosis, Trichomonas vaginitis, candida, non-infectious vaginitis, inflammatory vaginitis as well as sepsis and psychiatric diseases, preferably schizophrenia, Parkinsonism, bipolar disorder, depression or autism.

According to another preferred embodiment, the peptide is present as dimer, preferably a homodimer. The dimers are preferably bound via a covalent bond, suitably a disulfide bond.

Dimerization of peptides is known in the art. The chemistry currently used for peptide dimerization involves chemoselective reactions between unprotected peptides. Examples are the formation of the following bonds, e.g., Cys-maleimide thioethers, disulfides or triazoles.

It is to be understood that the before-mentioned features and those to be mentioned in the following cannot only be used in the combination indicated in the respective case, but also in other combinations or in an isolated manner without departing from the scope of the invention.

The invention is now further explained by means of embodiments resulting in additional features, characteristics and advantages of the invention. The embodiments are of pure illustrative nature and do not limit the scope or range of the invention.

The features mentioned in the specific embodiments are also features of the invention in general, which are not only applicable in the respective embodiment but also in an isolated manner in the context of any embodiment of the invention.

### BRIEF DESCRIPTION OF THE DRAWINGS

The invention is also described and explained in further detail by referring to the following drawings:

FIG. 1A shows the results of experiments for proving that HD-6 nanonet formation is not affected by duodenal fluid illustrating the chromatograph of HD-6 incubated with duodenal fluid after reduction with 2 mM TCEP. Only the oxidized and reduced full-length peptides were detected, due to their retention time with their m/z graphs and their 2-, 3—, 4-, 5- and 6-fold protonated ions and neutral masses.

FIG. 1B shows incubated reduced beads with 200 μg/ml HD-6 or 0.01% HAc (control) and duodenal fluid or 0.9% NaCl (control). The nanonet formation was not affected as these nets look the same as HD-6 with 0.9% NaCl. Magnification bar=0.2 μm.

FIG. 2A-FIG. 2C show the results of experiments where incubation of HD-5 and duodenal fluid led to many different fragments. HD-5 was incubated with duodenal fluid after reduction with 2 mM TCEP. FIG. 2A shows the overview of the chromatogram from incubation of reduced HD-5 with duodenal fluid. All detectable fragments were marked in grey (a-m) and listed due to their retention time in FIG. 2B. The mass-to-charge (m/z) graphs of all identified fragments with the detected ions and their neutral masses. The peptides (a), (b), (c), (e), (i), (j), (k), (l) and (m) were chosen for synthesis and deeper investigation of their abilities. FIG. 2C Here the chosen fragments are listed (in grey) with their amino acid sequence and their distribution over the HD-5 sequence.

FIG. 3A-FIG. 3C shows the results of experiments proving that HD-5 fragments are antimicrobial active peptides against commensal bacteria: FIG. 3A shows a table summarizing the testing of different commensal bacteria due to their susceptibility to HD-5 fragments. In this heat map all bacteria are listed and the activity of the fragments in RDA against them. In the RDA 2 μg of the full length and 4 μg of each fragment were used. An inhibition zone greater than 5 mm was determined as high activity, between 2.5 and 5 mm as low activity and 2.5 mm were the diameter of the punched well and therefore no activity. FIG. 3B shows, in diagrams, the original data from FIG. 3A are placed with mean and standard deviation from at least three independent experiments.

FIG. 3C shows electron microscopy pictures investigating the mode of action of the different peptides: E. coli MC1000 was incubated with all different fragments and transmission electron microscopy was performed and the resulting phenotypes were analyzed. Magnification bars of all pictures are 0.5 μm except the full-length peptide, HD-5_{fl} (1 μm).

FIG. 4A-FIG. 4B shows the results of experiments proving the antimicrobial activity of the different fragments against pathogenic bacteria: FIG. 4A shows a table summarizing the testing of the antimicrobial activity of HD-5 fragments against pathogenic bacteria. A heat map system with high activity (inhibition zone in the RDA>5 mm), low activity (2.5 to 5 mm) and no activity (2.5 mm) was used. FIG. 4B Shows diagrams of the data of FIG. 4A with mean and standard deviation from at least three independent experiments.

FIG. 5A-FIG. 5B shows the results of experiments proving that HD5₁₋₉ containing cysteine and arginine substitutions show hardly any antimicrobial effects against E. coli and S. aureus mutants. The minimal inhibition concentration (MIC) of HD5₁₋₉ and its variants was determined against. FIG. 5A E. coli BW 25113 mutants and FIG. 5B S. aureus SA113 mutants due to the measurement of the optical density (OD₆₀₀) after 18 h. Results from three independent experiments with +/−SEM are represented.

FIG. 6 shows diagrams of experiments summarizing that reduction of HD5₁₋₉ and synthetic HD5₁₋₉ causes complete dissolving of its antimicrobial activity. The minimal inhibition concentration (MIC) was determined of (A) E. coli BW 25113 and (B) S. aureus SA113 with several concentrations of reduced and oxidized HD5₁₋₉ and dimer due to the measurement of the optical density after 18 h. Afterwards bacteria were plated out to confirm MIC. Results from three independent experiments with +/−SEM are represented.

FIG. 7A-FIG. 7C shows diagrams of experimental data showing that almost no reduction in the metabolic activity of HD5₁₋₉ treated cells was observed. To analyze the metabolic activity of HD5₁₋₉ and dimer treated cells, a WST-1 assay was performed. Cell lines were stimulated either with HD5₁₋₉ or HD5₁₋₉ dimer with concentrations between 3.123-100 μM and incubated for 24 h or 48 h. The activity is normalized to the negative control. As positive control, cells were treated with 2% Triton X-100 while treatment with 0.01% acetic acid was used as negative control. The results show the mean with +/−SEM of three independent experiments (FIG. 7A, FIG. 7B, and FIG. 7C).

FIG. 8A-FIG. 8C shows the anti-microbial mode of action of HD5₁₋₉. Cell wall damages induced due to HD5₁₋₉ differ between E. coli ATCC 25922 and S. aureus SA113. In order to detect bacterial cell damages caused by HD5₁₋₉, a flow cytometry analysis was performed. 1.5×10⁶ E. coli ATCC 25922 and S. aureus SA113 were incubated with different concentrations of HD5₁₋₉ (6.25 μM, 12.5 μM and 50 μM) for 1 h. Either FIG. 8A propidium iodide or the FIG. 8B membrane-sensitive DiBAC₃(3) dye was used to stain bacteria. As positive control, 12.5 μM hBD3 was used while untreated cells function as negative control. Results from three independent experiments with +/−SEM are represented. FIG. 8C Transmission electron microcopy was performed to evaluate morphological changes of HD5₁₋₉ (200 μg/ml) treated E. coli MC1000. For comparison, the full length HD5 (HD5_{fl}) was used while treatment with 0.01% acetic acid (HAc) functions as negative control. Bars: Upper left panel: 1 μm; Upper right and lower left: 0.5 μm; lower right panel: 2 μm.

FIG. 9A-FIG. 9B shows results of investigations and experiments regarding Akkermansia in feces and susceptibility to HD-51-9 treatment: FIG. 9A The amount of Akkermansia sp. in feces samples (day 0, 7, 14) collected from mice treated for 7 days with HD-51-9 or PBS is increased in HD-51-9 treated animals compared to PBS treated ones (linear mixed-effect model; p=0.075). Here the mean is shown with the 80% confidence interval from n=6 per group. In FIG. 9B it was tested if Akkermansia muciniphila is susceptible to HD-51-9 treatment. FIG. 9B shows the growth rate compared to an untreated control in % after 72 hours of incubation at 37° C. in an anaerobic jar. A MIC from HD-51-9 against of Akkermansia could not be detected in this assay. The graph shows the mean with standard deviation of n=3.

FIG. 10 shows the pro- and anti-inflammatory immune response of HD5₁₋₉ stimulated human PBMCs. Human peripheral blood mononuclear cells (PBMCs) were isolated and stimulated with 10 μg/ml LPS (S. typhimurium) and different concentrations of HD5₁₋₉ followed by an incubation of 24 h. Supernatant of PBMCs was used to quantify the number of produced cytokines by a multi-analyte kit (LEGENDplex). The following cytokines were evaluated: Pro-inflammatory cytokines (A) TNF-α (B) IFN-γ (C) IL-1β (D) IL-6 (E) IL-8 and anti-inflammatory cytokine (F) IL-10. As negative control untreated cells were used and the cytokine concentration normalized to the negative control. The results show the mean with +/−SEM of three independent experiments. For statistical analysis a Kruskal-Wallis Test was performed. p>0.05=ns; p≤0.05=*; p≤0.01=**; p≤0.001=***; p<0.0001=****.

FIG. 11A-FIG. 11C shows that the toxicity profile of HD5₁₋₉ and its dimerized form displays no cytotoxicity effects on human cell line. The FIG. 11A metabolic activity and FIG. 11B cytotoxicity of HD5₁₋₉ and dimer was determined using a WST-1 assay and LDH assay. HT29-MTX-E12 cells were stimulated either with HD5₁₋₉ or HD5₁₋₉ dimer with concentrations between 3.123-100 μM and incubated for 48 h. The activity was either normalized to the negative or positive control. As positive control, cells were treated with 2% Triton X-100 while treatment with 0.01% acetic acid was used as negative control. The results show the mean with +/−SEM of three independent experiments. FIG. 11C illustrates the hemolytic activity of HD5₁₋₉ as analyzed. Red blood cell suspension was incubated with different concentrations of HD5₁₋₉. Hemolytic activity was normalized to the hemolytic activity of 0.1% Triton X-100. The experiments were carried out in duplicates.

FIG. 12A-FIG. 12C shows additional tox data. Almost no cytotoxic effects of TR146 cells were observed after peptide treatment. An LDH assay was performed in order to evaluate cytotoxic effects of HD5₁₋₉ and dimer. Cell lines were stimulated either with HD5₁₋₉ or HD5₁₋₉ dimer with concentrations between 3.123-100 μM and incubated for 24 h or 48 h. The activity was normalized to the positive control. As positive control, cells were treated with 2% Triton X-100 while treatment with 0.01% acetic acid was used as negative control. The results show the mean with +/−SEM of three independent experiments. Cytotoxic effects of TR146 cells were evaluated for FIG. 12A HD5₁₋₉ after 24 h and 48 h as well as FIG. 12B HD5₁₋₉ dimer after 24 h and 48 h. Additional, the cytotoxicity of FIG. 12C HD5₁₋₉ and dimer was determined of HT29-MTX-E12 cells after 24 h.

FIG. 13 shows the overall fecal microbial community. PCoA of weighted (A) and unweighted (B) UniFrac distances using all mice (n=12 per group) after 1 week of treatment comparing HD5 fl with HD5₁₋₉.

FIG. 14 shows the overall small intestinal microbial community. PCoA of weighted (A) and unweighted (B) UniFrac distances using all mice sacrificed week 1 (n=6 per group) after 1 week of treatment comparing HD5 fl with HD5₁₋₉.

FIG. 15 shows the bacterial genera (A) differently affected by full length and fragmented (B) HD-5 treatment. Linear mixed model adjusted for cages on fecal microbiota day 0, 7 and 14 using all mice (note that n=12 per group day 0 and 7 and n=6 per group day 14). Only significantly different genera presented.

FIG. 16: Antimicrobial activity of HD5₁₋₉ against E coli ATCC 25922 and E. coli BW 25113 as well its LPS mutants. (A) Cell wall construction of E. coli BW 25113 mutants. E. coli ATCC 25922 contains the full-length LPS whereas the O-antigen is missing in E. coli BW 25113. The E. coli BW 25113 mutant ΔwaaG miss the outer core in contrast to ΔwaaY lacking some phosphate residues in the inner core. The last mutant ΔwaaP contains an outer core but no phosphate residues in the inner core. (B) The minimal inhibition concentration (MIC) of HD5₁₋₉ was determined against E coli ATCC 25922 and E. coli BW 25113 mutants with different peptide concentrations due to the optical density after 18 h. Results from at least two independent experiments with Mean +/−SEM are represented.

FIG. 17A-FIG. 17B: Antimicrobial activity of HD5₁₋₉ against S. aureus SA113 as well as its cell wall mutants. FIG. 17A: S. aureus SA113 cell wall mutants were used to analyze charge-dependent antimicrobial effects of HD5₁₋₉. S. aureus mutant ΔdltA lacks D-Alanine leading to a more negative charge of the peptidoglycan layer. Similar characteristics possess the mutant ΔmprF missing L-Lysin causing a negative charge of the cell membrane. The S. aureus mutant ΔtarH contains additional teichoic acid resulting in a strengthening of the peptidoglycan layer. FIG. 17B: The minimal inhibition concentration (MIC) of HD5₁₋₉ was determined against S. aureus SA113 and mutants with different peptide concentrations due to the optical density after 18 h. Results from two independent experiments with +/−SEM are represented.

FIG. 18: Antimicrobial effects of HD5₁₋₉ differentiate compared to the dimer form against Gram-negative bacteria. The minimal inhibition concentration (MIC) of HD5₁₋₉ and HD5₁₋₉-dimer was determined against different Salmonella species with different peptide concentrations due to the optical density after 18 h. Results from at least two independent experiments with Mean+/−SEM are represented.

FIG. 19: Antimicrobial effects of HD5₁₋₉ differentiate compared to the dimer form against Gram-positive bacteria. The minimal inhibition concentration (MIC) of HD5₁₋₉ and HD5₁₋₉-dimer was determined against S. aureus ATCC25923 and the clinical isolate S. aureus USA300 with different peptide concentrations due to the optical density after 18 h. Results from two independent experiments with +/−SEM are represented.

FIG. 20: Reduced HNP-4 is digested by trypsin. (a) Displays an overview of the chromatogram from an incubation of reduced HNP-4 with trypsin after reduction with 2 mM TCEP. All detectable fragments were marked in red or grey (a-j) and listed due to their retention time. The full length peptide is marked as (i) and the fragment HNP-4₁₋₁₁ (d).

FIG. 21: HNP4-derivates display high antimicrobial activity against commensal and pathogenic bacteria. Antimicrobial potential was analyzed of the identified fragment and its modified version against commensal and pathogenic bacteria using a RDA. Showing a heat map, an inhibition zone greater than 5 mm was determined as highly active, between 2.5 and 5 mm as low active, while a diameter of 2.5 mm (diameter of the punched well) was marked as not active. The heat map is based on at least three independent experiments.

FIG. 22A-FIG. 22C: HNP-41-11 and HNP-41-11mod show only minor cytotoxic and hemolytic activity at high concentrations. We investigated the cytotoxic activity of HNP-4₁₋₁₁ and HNP-4_{1-11mod} against FIG. 22A CaCo2/TC7 or FIG. 22B HT29 MTX E 29 cells. We seeded 1500 cells per well and treated them after 24 hours with different peptide concentrations. Living cells were determined after 96 hours treatment using a CellTiter Glo2.0 assay. FIG. 22C: Hemolytic activity on human erythrocytes of the peptides compared to 0.1% Triton-X treatment.

### DETAILED DESCRIPTION

### Material and Methods

Bacterial strains

A. baumannii 4-MRGN, K. pneumoniae 4-MRGN, P. aeruginosa ATCC27853, E. faecium 475747, B. longum, L. fermentum, L. salivarius and S. salivarius salivarius were obtained as clinical isolates from the Robert-Bosch-Hospital (Stuttgart, Germany). Akkermansia muciniphila, B. subtilis 168trpC and S. aureus USA300 were received from the Institut für Mikrobiologie und Infektionsmedizin (Tübingen, Germany). B. adolescentis Ni3,29c, B. breve were provided by Ardeypharm. B. vulgatus DSM1447 was obtained from DSMZ and L. rhamnosus were provided by InfektoPharm (Heppenheim, Germany). Escherichia coli ATCC 25922 was obtained from Deutsche Sammlung von Mikroorganismen und Zellkulturen GmbH (Bonn, Germany). Clinical isolate of Salmonella species as well as Staphylococcus aureus USA300, Staphylococcus aureus ATCC 25923, Staphylococcus aureus SA133 and its mutants were provided by the Institute of Medical Microbiology and Hygiene Tübingen, Germany. Escherichia coli BW25113 as well as its mutants were obtained from the Interfaculty Institute for Microbiology and Infection Medicine, Tübingen, Germany. Peptides For all experiments oxidized peptides HD-5 and HD-6 (Peptide Institute, Osaka, Japan) were used. All fragments, i.e. HD-5₁₋₉ and HNP-4₁₋₁₁, HD-5₁₋₁₃, HD-5₁₋₂₈, HD-5₇₋₃₂, HD-5₁₀₋₃₂, HD-5₁₄₋₃₂, HD-5₁₀₋₂₇ and HD-5₂₆₋₃₂ (all peptides of the invention), were synthesized by EMC microcollections GmbH (Tübingen, Germany). All peptides were dissolved in 0.01% acetic acid (HAc) in similar concentrations.

The following sequences (peptides according to the invention) were tested (N→C-terminus):

HD-5₁₋₉:
(SEQ ID No. 1)
ATCYCRTGR
HD-5₁₋₉ᵣₑᵥ:
(SEQ ID No. 2)
RGTRCYCTA
HD-5_{1-9mod}:
(SEQ ID No. 5)
Ac-atcycrtGr-NH₂
HNP-4₁₋₁₁:
(SEQ ID No. 3)
VCSCRLVFCRR
HNP-4₁₋₁₁ᵣₑᵥ:
(SEQ ID No. 4)
RRCFVLRCSCV
HNP-4_{1-11mod}:
(SEQ ID NO. 6)
Ac-vcscrlvfcrr-NH₂

### Collection of Duodenal Fluid During Gastroscopy.

The human duodenal fluid was collected during a routine gastroscopy from three healthy individuals. The duodenum was washed with 0.9% NaCl solution, which was recollected. Patients gave their written and informed consent after they were informed. The sample collection had been previously approved by the Ethical Committee of the University Hospital of Tuebingen, Germany.

### Screening for Fragments of HD-5 and HD-6 Using LC/MS

2.5 μg of HD-5 or HD-6 were incubated in 50 mM NH₄HCO₃ buffer (pH 8.0) (Fluka) with 2 mM tris(2-carboxyethyl) phosphine for 15 minutes at 37° C. Afterwards human duodenal fluid was added and incubated for additional 30 minutes at 37° C. At last, formic acid and acetonitrile was added in a final concentration of 0.5% and 10%, respectively, and analyzed the samples by mass spectrometry. Mass spectrometry was performed as a LC/MS system using an Agilent 1200 series HPLC with an Agilent Advanced Bio Peptide Map (2.1×150 mm, 2.7 μm) column with a flow of 0.4 ml/min at 55° C. column temperature and a 6540 UHD Q-TOF LC/MS system (Agilent) for mass analysis. The samples were separated by a gradient of acetonitrile in 0.1% formic acid. The gradient started at 2% acetonitrile for 4 minutes and then increased during 35 minutes to 45%. Mass spectrometric analyses were performed in single MS mode from 100 to 3400 m/z with positive ion polarity and were analyzed by Agilent MassHunter Quantitative Analysis B 06.00 software.

### Scanning-Electron-Microscopy

Scanning-Electron-Microscopy was performed as previously described. Briefly, Protein A coated beads (Spherotech Inc.) were incubated with reduced HD-6 (200 μg/ml) in 10 mM sodium phosphate buffer with 1% (w/v) TSB for 1.5 hours at 37° C. to allow net formation. We subsequently incubated the whole sample with duodenal fluid for additional 30 minutes at 37° C. As a control 0.01% acetic acid (HAc) was used. Beads were centrifuged and fixed in Karnovsky's reagent. The samples were washed with PBS and additional fixed with 1% OsO₄ in H₂O. Then, they were dehydrated to 100% ethanol and critical point dried from CO₂ and analyzed by scanning electron microscopy at the Max Planck Institute for Developmental Biology (Tuebingen, Germany).

### Transmission Electron Microscopy

Transmission electron microcscopy experiments were performed as previously described¹¹. 6×10⁸ cfu E. coli MC1000 was incubated with 200 μg/ml of each peptide for 2 hours. Bacteria were fixed in Karnovsky's fixative, embedded in agarose, coagulated, cut in small blocks and fixed again in Karnovsky's solution. After post-fixation and embedding in glycid ether blocks were cut using an ultra-microtome. Sections (30 nm) were mounted on copper grids and analyzed using a Zeiss LIBRA 120 transmissions electron microscope.

### Radial Diffusion Assay

Antimicrobial activity of all peptides was tested with a modified radial diffusion assay from Lehrer et al¹² Shortly described, log-phase bacteria grew (anaerobic bacteria with AnaeroGen, Oxoid in anaerobic jars) in liquid tryptic soy broth (TSB) (Becton Dickinson). After several wash steps in 10 mM sodium phosphate buffer pH 7.4, 4×10⁶ cfu/ml was used per assay. To measure the antibacterial effect of the identified peptide fragments, the bacteria were incubated in 10 mM sodium phosphate (pH 7.4) containing 0.3 mg/ml TSB powder and 1% (w/v) low EEO-agarose (Applichem). Peptide fragments were then pipetted into punched wells and allowed to diffuse for 3 hours at 37° C. After that a nutrient rich gel with 6% TSB (w/v) and 1% agarose in 10 mM sodium phosphate buffer was poured on top of the first gel. After 24 hours the inhibition zones were measured. We used 0.01% acetic acid as a negative control, which did not show inhibition zones greater than the diameter of the punched well. All experiments were carried out at least three times.

### Turbidity Broth Assay

The tested bacteria were incubated overnight in 1x TSB broth, centrifuged and washed with 10 mM sodium phosphate buffer containing 1% (w/v) TSB broth.

5×10⁵ cfu/ml bacteria were mixed with different peptide concentrations in 10 mM sodium phosphate buffer with 1% (w/V) TSB (final volume 100 μl) and incubated for 2 hours at 37° C. Afterwards we added 100 μl 2×TSB broth and we measured the optical density at 600 nm (Spark 10M, Tecan, Austria). Bacterial growth was monitored for 12 hours, growing at 37° C. with shaking during the measurements each 30 minutes, except for Akkermansia muciniphila which were incubated at 37° C. in an anaerobic jar and growth was measured after 72 hours.

Bactericidal activity of the E. coli and S, aureus strains in the cell wall target experiments as well as the HD5 dimer experiments was assessed as described previously. Log-phase bacteria were collected by centrifugation (2500 rpm, 10 min, 4° C.), washed twice with 10 mM sodium phosphate buffer containing 1% (w/v) TSB broth and the optical density at OD600 nm (OD600=0.1) was determined. Approximately 5 ×10⁵ CFU/ml bacteria were incubated with serial peptide concentrations (1.17-150 μM) in a final volume of 100 μl in 10 mM sodium phosphate buffer containing 1% (w/V) TSB broth for 2 hours at 37° C. After incubation, 100 μl of 6% TSB (w/v) were added and absorbance was measured at 600 nm (Tecan, Switzerland) and monitored for 18 hours. Afterwards, 100 μl per well were plated on LB-plates to determine the numbers of viable bacteria microbiologically. Bactericidal activity is expressed as the LC99.9, the lowest concentration that killed ≥99.9% of bacteria. The experiment was repeated at least three times independently.

### Cell Cytotoxicity Assay

CaCo2/TC7 (X,X) and HT29 MTX E29 (X,X) were seed in a 96 well plate in 90 μl media (1500 cells/well) and incubated at 37° C. for 24 hours. Afterwards peptide treatment with different concentrations started (volume of 10 μl solved in 0.01% acetic acid) and cells were incubated for 96 hours. Untreated and 1% Triton-X treated cells were used as controls. After the incubation we added 100 μl of CellTiter Glo2.0 solution and started our measurement protocol. The measurement was carried out in a Spark 10M (Tecan), starting with 12 minutes continuously shaking followed by the luminescence measurement with an integration time from 1 second per well. Experiments were carried out in duplicates.

### Hemolytic Assay

Hemolytic activity was measured after an existing protocol¹³. We obtained blood from two voluntary donors (sample collection had been previously approved by the Ethical Committee of the University Hospital of Tübingen, Germany) and 1 ml blood was washed with PBS two times. Afterwards we centrifuged the blood at 1000 g and performed a 1% (v/v) blood suspension in PBS. The blood suspension was incubated with different peptide concentrations (final concentration 0.5%) for one hour at 37° C. Then the samples were centrifuged at 1000 g for 10 minutes and the supernatant was collected and measured at 414 nm. The hemolytic activity was relative determined to the hemolytic activity of 0.1% Triton X-100. These experiments were carried out in duplicates.

### In Vivo Microbiota Analysis

To assess proof of concept of a functional impact on microbiota composition, HD-5₁₋₉ was administered to 9 weeks old healthy chow fed male mice housed in groups of 3 per cage. Mice were acclimatized for 3 weeks prior to the experiment start and stratified into experimental groups based on average body weight per cage, ensuring equal weight distribution between groups. In more detail, wildtype C57BL/6J mice were treated by oral gavage for 7 days with 7.19 μg/mouse HD-51-9 administered in 100 μL PBS solution. Control mice were treated with equal volume PBS. Initially, a 7-day experiment was performed with 6 mice per group. Based on these results, a new study was designed, also including 6 mice per group, to study the temporal impact of gut microbial modulations. In this study, a 7 days wash period was included, after 1 week of oral gavage. Fresh feces samples were collected from individual mice at day 0, 7 and 14 (treated and control group n=6 in each) at 9AM the same time body weight was measured. At day 14, mice were euthanized and the small intestine content was collected.

Bacterial DNA was extracted from snap-frozen feces collected at day 0, 7 and 14, and content of small intestine at necropsy by the NuceloSpin 96 soil kit (Macherey-Nagel) following the manufacturer's instructions. BGI, Europe performed the subsequent library preparation and DNA sequencing using in-house standard operating procedures. In brief, 30 ng of bacterial DNA per sample was PCR amplified using the primers: 515F: GTGCCAGCMGCCGCGGTAA (SEQ ID No. 7), 806R: GGACTACHVGGGTWTCTAAT (SEQ ID No. 8) with Illumina adapters targeting the V4 16S rDNA region. PCR products were then purified with AmpureXP beads (AGENCOURT) to remove unspecific products. The average molecule length was determined by Agilent 2100 bioanalyzer (Agilent DNA 1000 Reagents). DNA quantification was evaluated by real-time quantitive PCR (EvaGreen™) before pair end sequencing on a HiSeq2500 system.

Processing and quality control of reads was performed using the R package DADA2, version 1.4.0¹⁴ and forward and reverse primers were trimmed off from reads. Next, all reads containing remaining uncalled bases or more than two expected errors were removed. Afterward, the parameters of the DADA2 error model were learned from a random subset of 1 million reads. This error model was then used to denoise all sequences; i.e., to infer the ASVs. Denoised reads (ASVs) were then merged and read pairs with one or more conflicting bases between the forward and reverse read were removed. ASVs shorter than 251 and longer than 254 bases were discarded. Chimeric sequences were then detected and removed using the function “removeBimeraDenovo.” Finally, reads (ASVs) were classified from the kingdom to the genus level using the Silva reference 16S rRNA gene database, version 132 resulting in the construction of an ASV table with read counts of all ASVs in all samples.

All animal protocols were conducted according to guidelines set out by the Laval University Animal Care and Handling Committee. C57BL/6J male mice (Jackson Laboratories, Bar Harbor, ME) were housed in a pathogen-free, temperature-controlled environment under a 12:12 hour light-dark cycle and fed ad libitum standard rodent chow diet (Harlan Teklad T-2018) for the 5 weeks of accommodation in our vivarium (3 weeks of acclimatization and 2 weeks of experimental protocol).

### Statistical Analysis

Apart from microbiome analyses, all data were analyzed with GraphPad Prism 7. Values of p<0.05 were considered as statistically significant. All results are depicted as mean and their ±standard deviation, with their standard error of the mean or as 80% confidence interval, as indicated in the figure legend. Bioinformatical analysis was carried out using R Studio (R version 3.4.2 and R Studio version 1.0.136) and packages phyloseq 1.22.3¹⁵, metagenomeSeq 1.20.0¹⁶, vegan 2.4-4¹⁷, Ime4 1.1-15, and ggplot2 2.2.1¹⁸. For our mice studies we used cage-adjusted p-values.

### Software

For the in silico digest analysis the ExPASy PeptideMass tool from the SIB Bioinformatics Resource Portal (https://web.expasy.org/peptide_mass/) was used.

### Results

Natural human duodenal fluid digests HD-5, while nanonet forming HD-6 is protease resistant

Since Paneth cell defensins can be reduced by the natural occurring thioredoxin system, their susceptibility to a proteolytic digest was investigated. Before the experimental procedure was started, the possible fragmentation of HD-5 and HD-6 by intestinal proteases was investigated. The PeptideMass module of ExPASy (SIB Bioinformatics Resource Portal) was accordingly used to perform in silico digests of HD-5 and HD-6 with trypsin, chymotrypsin or a combination of both and allowed up to five missed cleavages. The possible fragments are listed in table 1 below on basis of their individual mass.

TABLE 1
In silico (normal letters) and ex vivo reality after duodenal
mucus incubation (in bold letters) digest of Paneth cell
HD-5 and HD-6 with trypsin or chymotrypsin or both in
combination, maximum of 5 missed cleavages and fragments
bigger than 500 Da. Determination of the different sequences
with the ExPASy PeptideMass module.
Fragments which can be identified with mass spectrometry
after incubation of the human peptides with human duodenal
mucus are bold. The first line in the table designates the
two full-length peptides, which could be identified too.

| SEQ | | | |
|---|---|---|---|
| ID | | missed | |
| No. | position | cleavages | sequence |
| | HD-5 | | |
| 9 | 1-32 | 5 | ATCYCRTGRCATRESLSGVCEISGRLYRLCCR |
| 10 | 1-29 | 4 | ATCYCRTGRCATRESLSGVCEISGRLYRL |
| 11 | 5-32 | 4 | CRTGRCATRESLSGVCEISGRLYRLCCR |
| 12 | 1-28 | 4 | ATCYCRTGRCATRESLSGVCEISGRLYR |
| 13 | 1-27 | 3 | ATCYCRTGRCATRESLSGVCEISGRLY |
| 14 | 7-32 | 4 | TGRCATRESLSGVCEISGRLYRLCCR |
| 15 | 1-26 | 2 | ATCYCRTGRCATRESLSGVCEISGRL |
| 16 | 5-29 | 3 | CRTGRCATRESLSGVCEISGRLYRL |
| 17 | 1-25 | 3 | ATCYCRTGRCATRESLSGVCEISGR |
| 18 | 5-28 | 5 | CRTGRCATRESLSGVCEISGRLYR |
| 19 | 10-32 | 3 | CATRESLSGVCEISGRLYRLCCR |
| 20 | 5-27 | 2 | CRTGRCATRESLSGVCEISGRLY |
| 21 | 7-28 | 3 | TGRCATRESLSGVCEISGRLYR |
| 22 | 5-26 | 1 | CRTGRCATRESLSGVCEISGRL |
| 23 | 7-27 | 3 | TGRCATRESLSGVCEISGRLY |
| 24 | 5-25 | 3 | CRTGRCATRESLSGVCEISGR |
| 25 | 14-32 | 2 | ESLSGVCEISGRLYRLCCR |
| 26 | 10-28 | 2 | CATRESLSGVCEISGRLYR |
| 27 | 7-25 | 2 | TGRCATRESLSGVCEISGR |
| 28 | 10-27 | 2 | CATRESLSGVCEISGRLY |
| 29 | 17-32 | 3 | SGVCEISGRLYRLCCR |
| 30 | 1-16 | 1 | ATCYCRTGRCATRESL |
| 31 | 14-28 | 1 | ESLSGVCEISGRLYR |
| 32 | 10-25 | 1 | CATRESLSGVCEISGR |
| 33 | 14-27 | 1 | ESLSGVCEISGRLY |
| 34 | 1-13 | 2 | ATCYCRTGRCATR |
| 35 | 17-29 | 2 | SGVCEISGRLYRL |
| 36 | 5-16 | 0 | CRTGRCATRESL |
| 37 | 14-25 | 0 | ESLSGVCEISGR |
| 38 | 17-27 | 1 | SGVCEISGRLY |
| 1 | 1-9 | 1 | ATCYCRTGR |
| 39 | 5-13 | 2 | CRTGRCATR |
| 40 | 17-26 | 0 | SGVCEISGRL |
| 41 | 26-32 | 1 | LYRLCCR |
| 42 | 27-32 | 2 | YRLCCR |
| 43 | 7-13 | 1 | TGRCATR |
| 44 | 1-6 | 0 | ATCYCR |
| 45 | 28-32 | 1 | RLCCR |
| 46 | 5-9 | 1 | CRTGR |
| | HD-6 | | |
| 47 | 1-32 | 3 | AFTCHCRRSCYSTEYSYGTCTVMGINHRFCCL |
| 48 | 3-32 | 5 | TCHCRRSCYSTEYSYGTCTVMGINHRFCCL |
| 49 | 1-29 | 5 | AFTCHCRRSCYSTEYSYGTCTVMGINHRF |
| 50 | 1-28 | 2 | AFTCHCRRSCYSTEYSYGTCTVMGINHR |
| 51 | 3-29 | 4 | TCHCRRSCYSTEYSYGTCTVMGINHRF |
| 52 | 3-28 | 5 | TCHCRRSCYSTEYSYGTCTVMGINHR |
| 53 | 8-32 | 2 | RSCYSTEYSYGTCTVMGINHRFCCL |
| 54 | 9-32 | 1 | SCYSTEYSYGTCTVMGINHRFCCL |
| 55 | 1-23 | 4 | AFTCHCRRSCYSTEYSYGTCTVM |
| 56 | 8-29 | 5 | RSCYSTEYSYGTCTVMGINHRF |
| 57 | 3-23 | 3 | TCHCRRSCYSTEYSYGTCTVM |
| 58 | 8-28 | 1 | RSCYSTEYSYGTCTVMGINHR |
| 59 | 9-29 | 4 | SCYSTEYSYGTCTVMGINHRF |
| 60 | 12-32 | 4 | STEYSYGTCTVMGINHRFCCL |
| 61 | 9-28 | 0 | SCYSTEYSYGTCTVMGINHR |
| 62 | 1-17 | 3 | AFTCHCRRSCYSTEYSY |
| 63 | 12-29 | 3 | STEYSYGTCTVMGINHRF |
| 64 | 12-28 | 2 | STEYSYGTCTVMGINHR |
| 65 | 16-32 | 3 | SYGTCTVMGINHRFCCL |
| 66 | 3-17 | 2 | TCHCRRSCYSTEYSY |
| 67 | 1-15 | 2 | AFTCHCRRSCYSTEY |
| 68 | 18-32 | 2 | GTCTVMGINHRFCCL |
| 69 | 3-15 | 1 | TCHCRRSCYSTEY |
| 70 | 16-29 | 2 | SYGTCTVMGINHRF |
| 71 | 16-28 | 1 | SYGTCTVMGINHR |
| 72 | 1-11 | 1 | AFTCHCRRSCY |
| 73 | 12-23 | 2 | STEYSYGTCTVM |
| 74 | 18-29 | 1 | GTCTVMGINHRF |
| 75 | 8-17 | 3 | RSCYSTEYSY |
| 76 | 18-28 | 0 | GTCTVMGINHR |
| 77 | 3-11 | 2 | TCHCRRSCY |
| 78 | 9-17 | 2 | SCYSTEYSY |
| 79 | 24-32 | 1 | GINHRFCCL |
| 80 | 8-15 | 2 | RSCYSTEY |
| 81 | 1-8 | 2 | AFTCHCRR |
| 82 | 16-23 | 1 | SYGTCTVM |
| 83 | 9-15 | 1 | SCYSTEY |
| 84 | 1-7 | 0 | AFTCHCR |
| 85 | 3-8 | 1 | TCHCRR |
| 86 | 12-17 | 1 | STEYSY |
| 87 | 24-29 | 0 | GINHRF |
| 88 | 3-7 | 0 | TCHCR |
| 89 | 18-23 | 0 | GTCTVM |
| 90 | 8-11 | 1 | RSCY |

In theory, both Paneth cell defensins seemed to be susceptible to proteases while HD-6 showed a tendency of more fragmentation as compared to HD-5 (Table 1). In a second step, the reducing agent tris(2-carboxyethyl) phosphine (TCEP) was used to reduce HD-5 or HD-6 and the peptides were challenged to natural occurring duodenal fluid, which is known to be proteolytically active. After performing a mass-spectrometric analysis a partly reduction with 2 mM TCEP for HD-6 was found, which is consistent with previous published work. Beside the two full-length forms

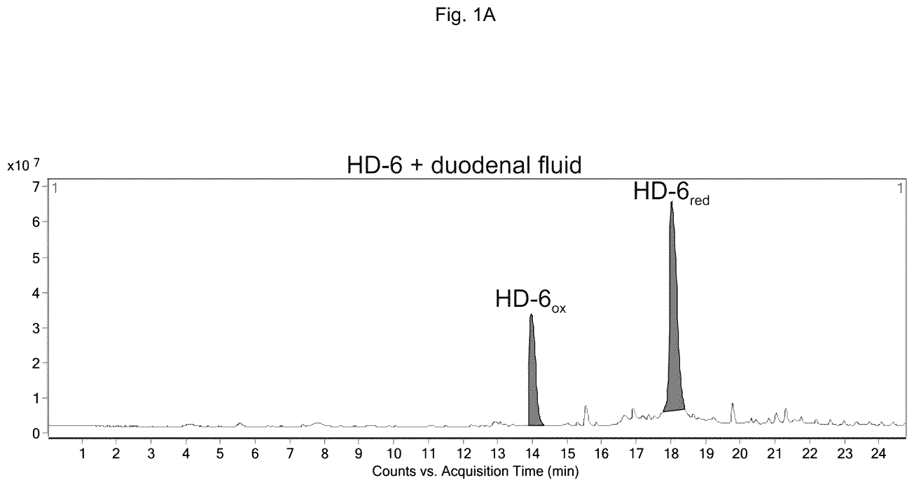

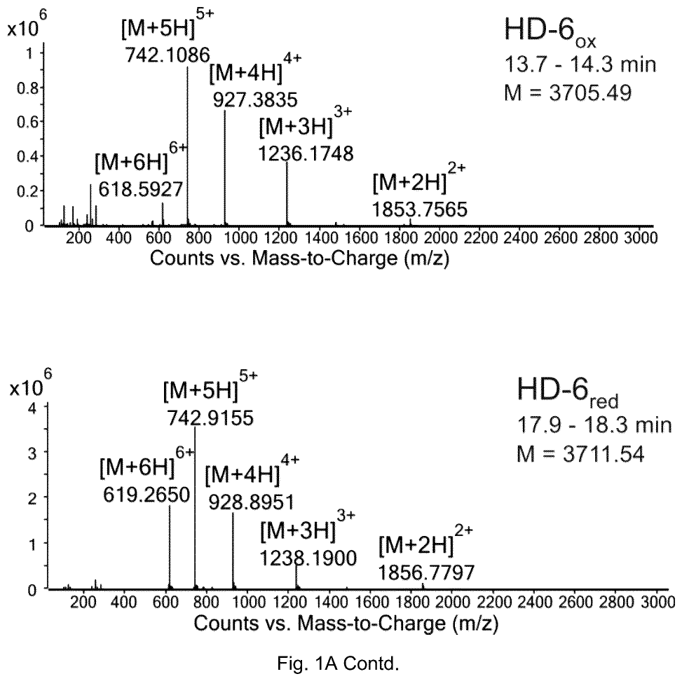

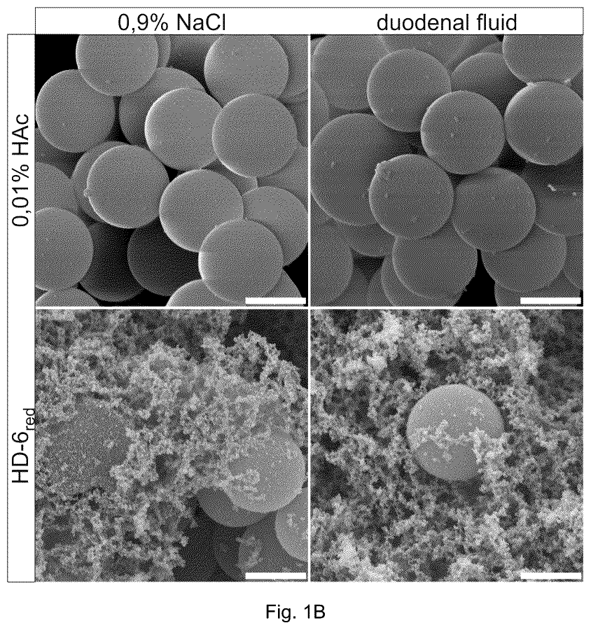

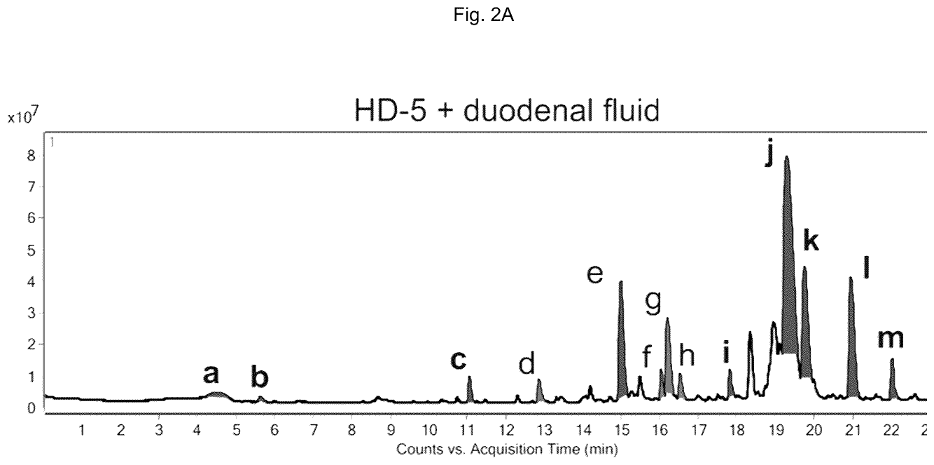

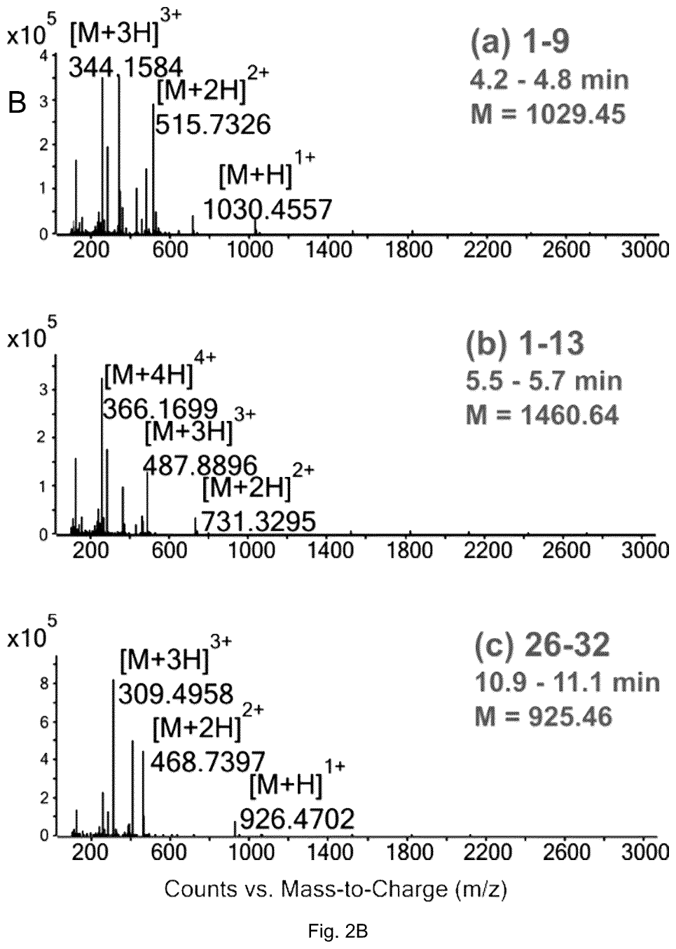

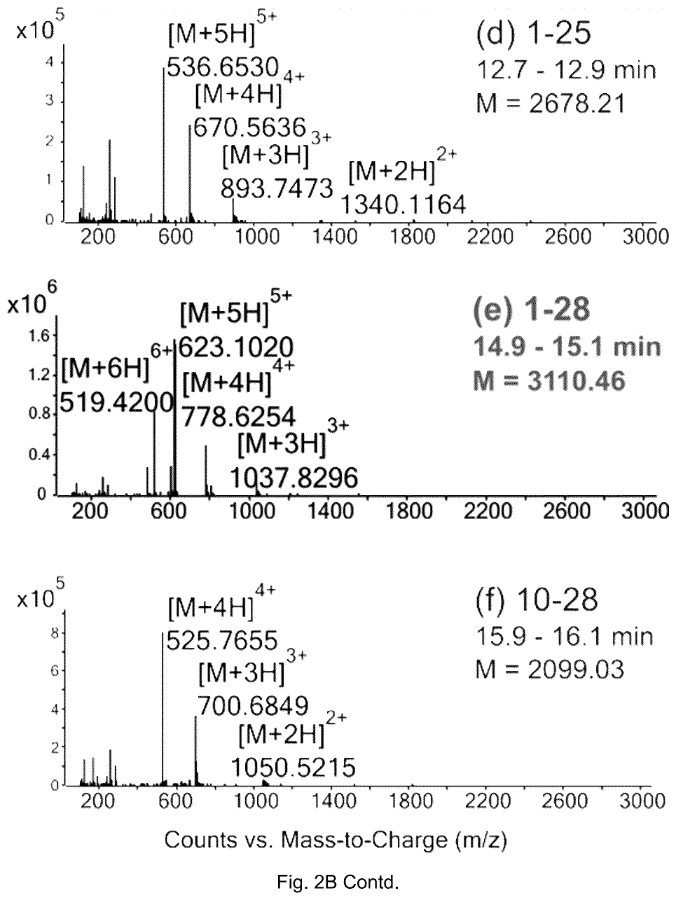

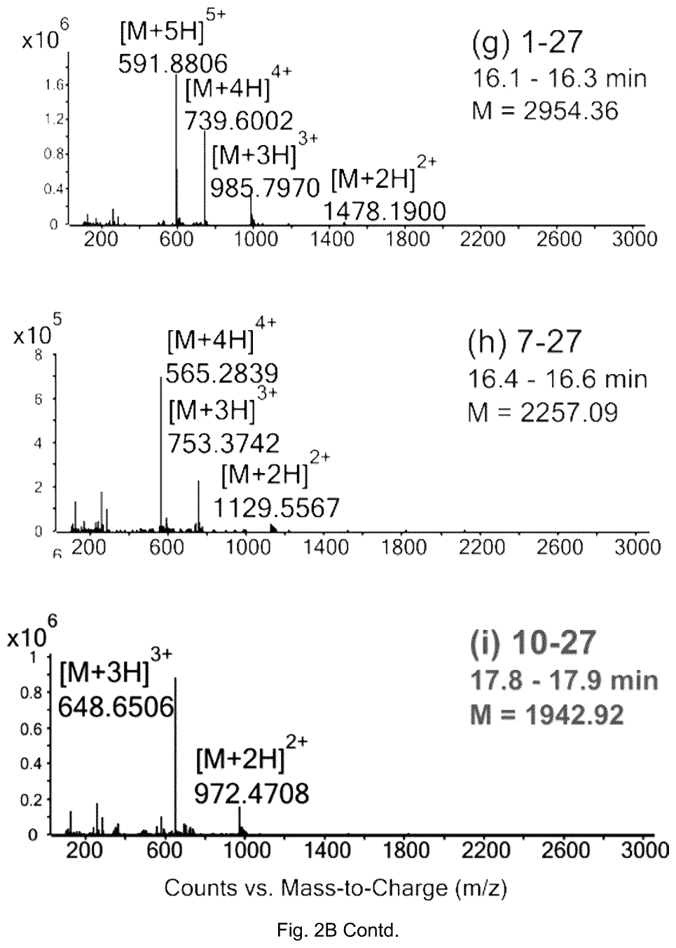

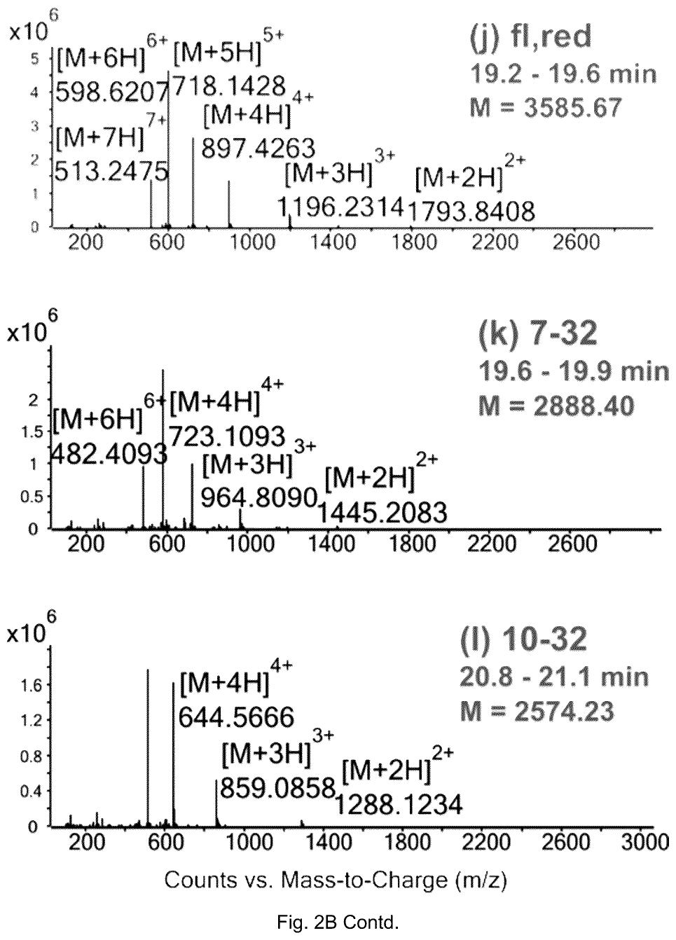

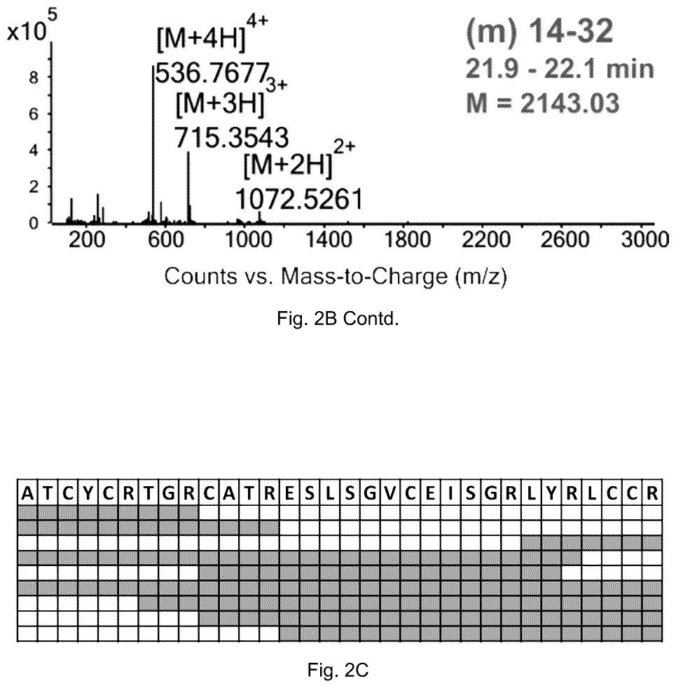

HD-6ₒₓ (expected: 3705.49 Da) and HD-6_{red} (3711.54 Da), surprisingly no other fragments were identified, identifiable by mass-to-charge-ratio (m/z) signals indicating 2-, 3-, 4-, 5-, 6-fold protonated ions. This surprising observation demonstrates that HD-6_{red} is protected against proteolytic digestion although the reason remains elusive, since proteolytic cleaving sites were bioinformatically predicted. It is known that both forms of HD-6 independent of its redox state (HD-6ₒₓ and HD-6_{red}) are able to form nanonets. Thus, it was hypothesized that net formation protects against protease degradation and thus might provide a mechanistic explanation for the observed peptide protection. To clarify if nanonet formation leads to a more stable structure, scanning electron microscopy from reduced HD-6 incubated with duodenal fluid was performed (FIG. 1). Consistent with this hypothesis, equal nanonets were observed independent of duodenal fluid incubation (FIG. 1B). Taken together, it appears that the nanonet formation at least contributes to prevent HD-6 from proteolytic digestion. Next, the second and more abundant Paneth cell defensin, HD-5, was studied in an identical experimental setup. After incubation with 2 mM TCEP and duodenal fluid, HD-5ₒₓ was below limit of detection (LOD), whereas HD-5_{red} was highly abundant. While HD-5 is not able to form nanonets, it was surprisingly observed that in contrast to HD-6, duodenal fluid had a differential effect on HD-5. Consistent with known protease cleaving sites, different fragments were identified which were not present before adding HD-5 (FIG. 2A, Table highlighted in bold letters). These fragments were analyzed and listed with their mass-to-charge ratios and the different observed protonated ions with their mass were shown for each fragment (FIG. 2B). The identified fragments were listed with their neutral mass and retention time, showing that the fragmentation of HD-5 with duodenal fluid led to abundant fragments derived from the entire peptide sequence. However, we still found detectable amounts of full length HD-5red suggesting that the proteolytic digestion was incomplete. It is known that Zn²⁺ can protect HD-5_{red} from proteolytic digestion, which was indeed confirmed in our setting (data not shown). In summary, it was shown that duodenal fluid surprisingly is affecting HD-5 and HD-6 differently and that changes of conditions in the local microenvironment have an impact on defensin fragmentation. Of note, HD-6 nanonet formation seems to protect against destruction of the reduced full length peptide and it is known that reduction of HD-6 unmasks antimicrobial activity. Therefore, reduction changes the activity of both Paneth cell defensins, but while HD-6 gains a direct antimicrobial activity, HD-5 is digested by intestinal proteases to form bioactive peptide fragments with potential bioactive antimicrobial activity.

### HD-5 Fragments are Antimicrobial Active in Radial Diffusion Assays

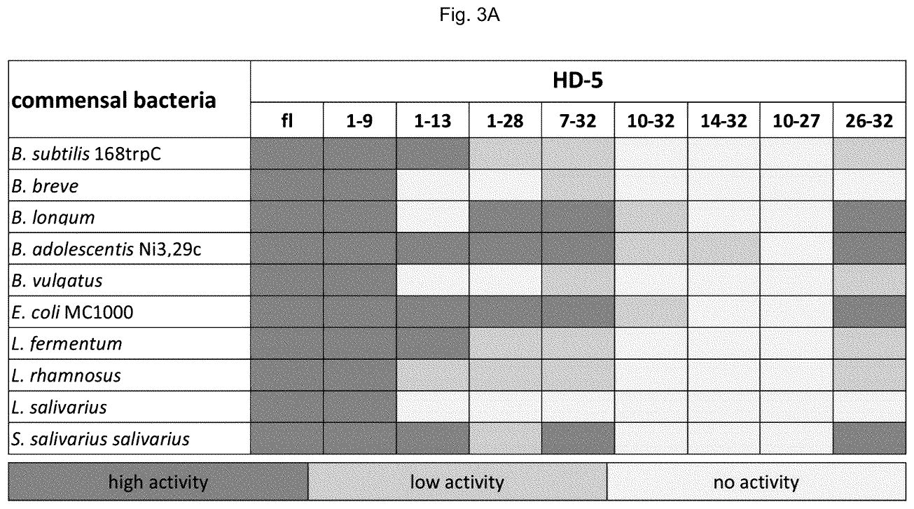

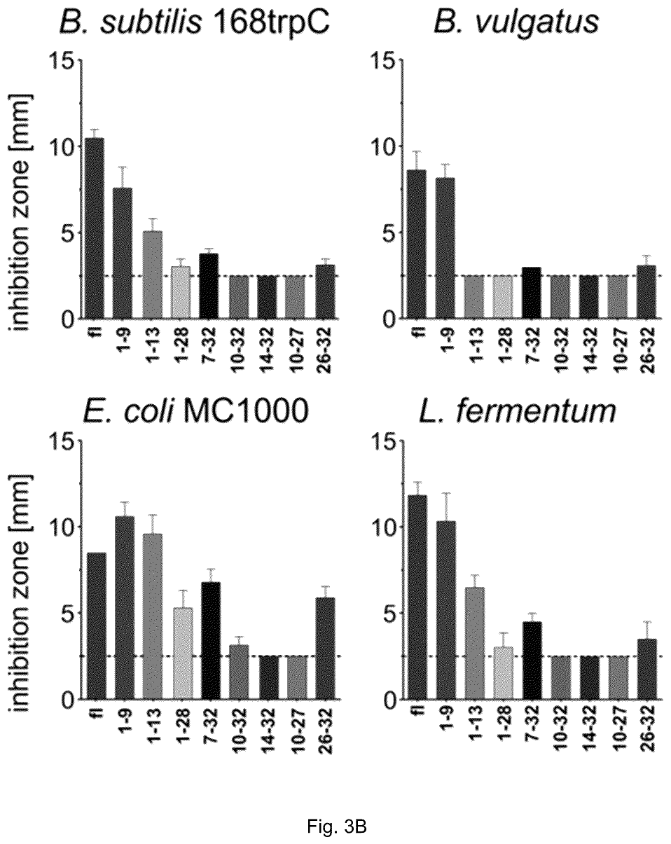

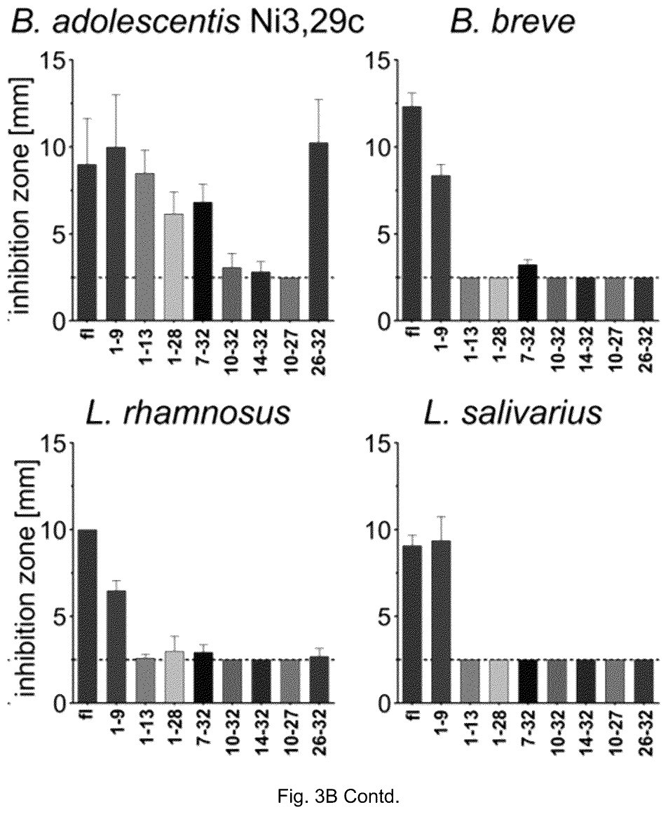

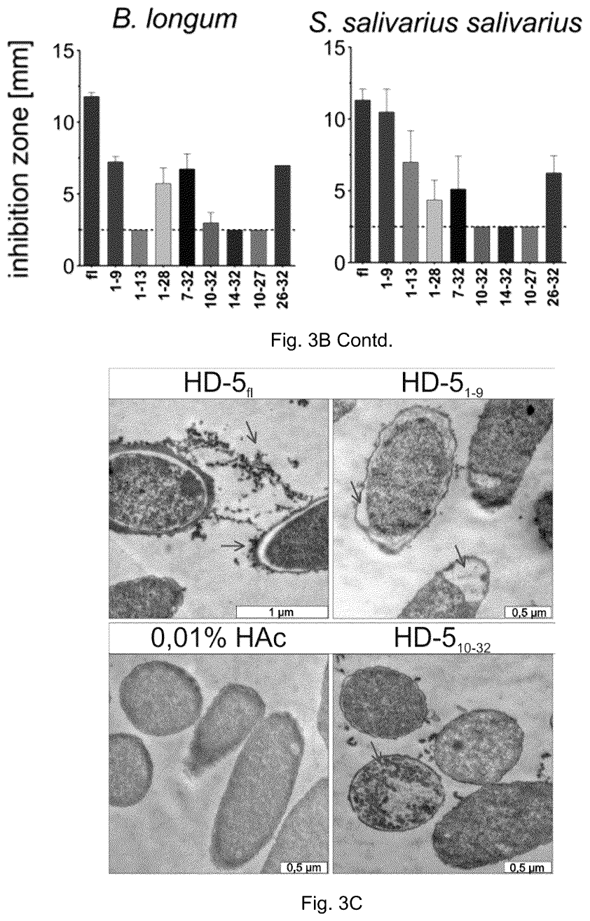

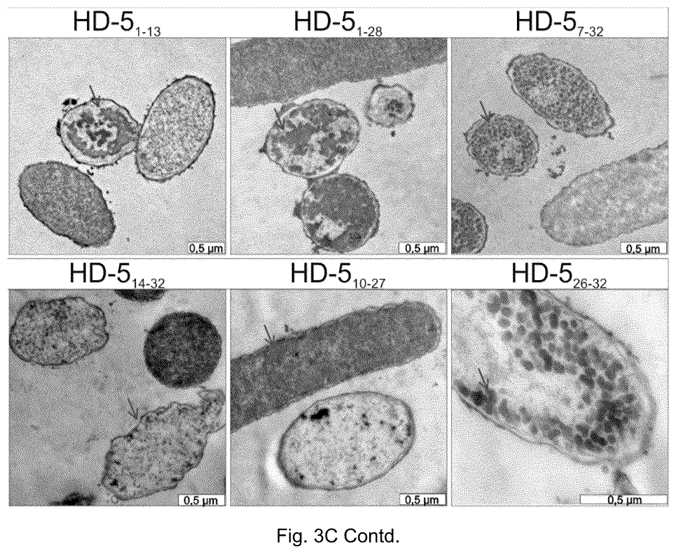

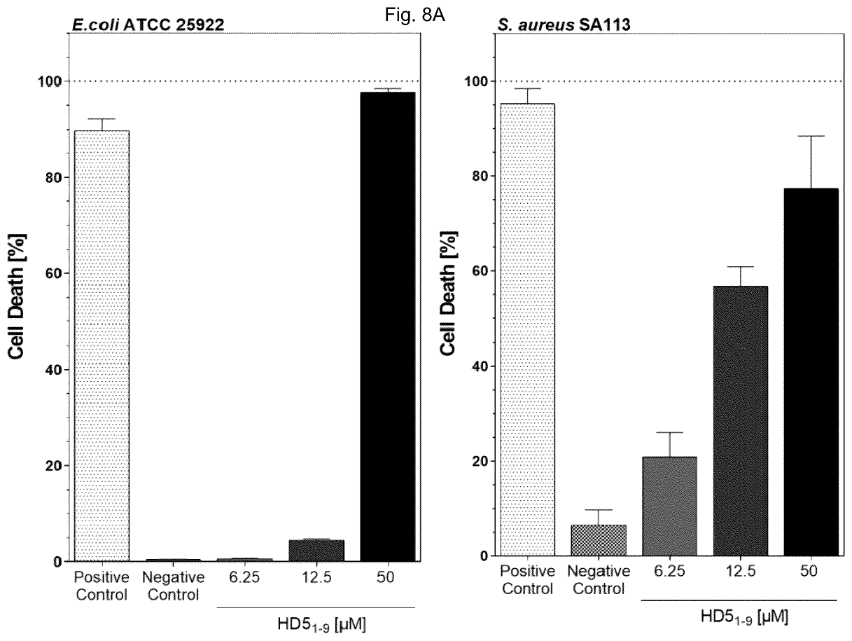

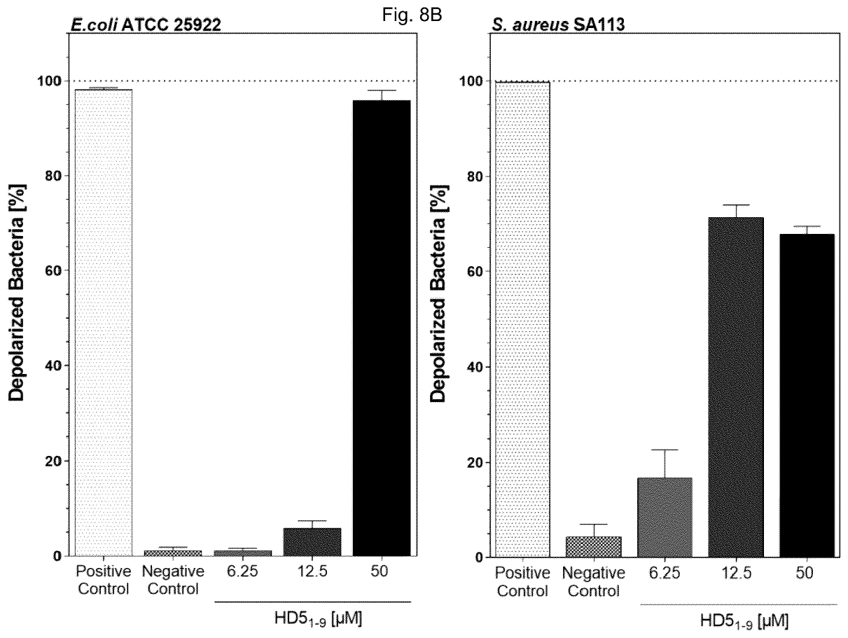

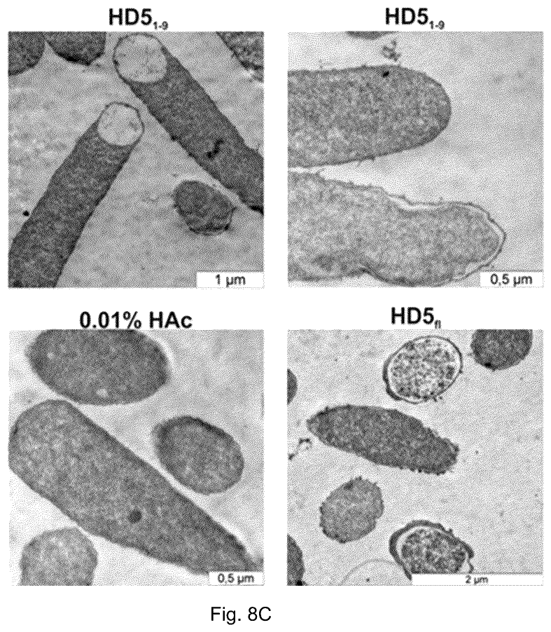

To test the antimicrobial activity from the HD-5 fragments, selected fragments were chemically synthesized to investigate antimicrobial functions in vitro (FIG. 2C). Several radial diffusion assays were performed with an amount of 4 μg of the different fragments and 2 μg of the full-length peptide against commensal gut and pathogenic bacteria. Interestingly and surprisingly, it was observed that most of HD-5 derived fragments to varying degree were antimicrobially active against commensal and pathogenic bacteria. Additionally, it was found that the different fragments exhibited distinct activity patterns (FIGS. 3A and B) against the applied bacteria strains. As expected the full length HD-5 displayed broad antimicrobial activity. Still and surprisingly HD-5₁₋₉ was identified as the most active peptide in terms of antimicrobial activity but also in terms of versatility since it was active against all tested bacteria. In contrast to HD-5₁₋₉, HD-5₁₀₋₂₇ did not exhibit measurable activity. Of note, HD-5₁₋₁₃, HD-5₁₋₂₈, HD-5₇₋₃₂ and HD-5₂₆₋₃₂ also exhibited antimicrobial properties, albeit to a lesser extent than HD-5₁₋₉. To further assess the resulting phenotype of peptide treated bacteria, E. coli MC1000 was incubated with all fragments and performed transmission electron microscopy (TEM) (FIG. 3C). It was observed that HD-5_{fl} treatment led to a detached inner membrane and small vesicular structures around the bacteria cell envelope (FIG. 8). Of interest, the bacteria surprisingly showed different typical phenotypes after incubation with the HD-5 different fragments which indicates discrepant modes of action. For HD-5₁₋₉ a detached inner membrane with additional big vacuole structures at one pole of the bacteria could be observed, while HD-5₁₋₁₃ and HD-5₁₋₂₈ treatment led to bigger aggregation inside the bacteria (FIG. 8). The findings indicate different kinds of bacterial phenotypes, and this variety suggests that minor sequence differences (e.g. HD-5₁₋₉ and HD-5₁₋₁₃) likely result in different mechanisms of host microbial interaction.

Of note and surprisingly, these observations showed that the antimicrobial activity of the different peptides did not apply to a certain group of bacteria. As an example the Bifidobacteria strains, B. adolescentis and B. longum were highly susceptible to the peptides, while B. breve was not. Collectively, the above-mentioned results demonstrate that a proteolytic digest of Paneth cell HD-5 leads to small, antimicrobial active fragments, which modulate commensal gut bacteria.

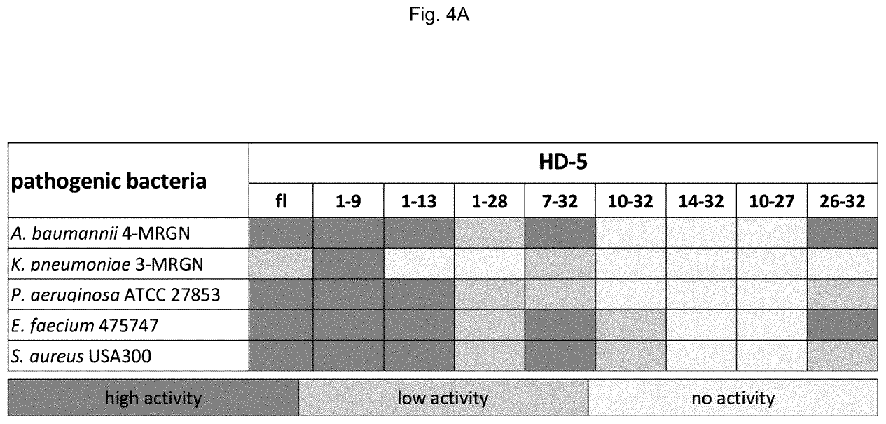

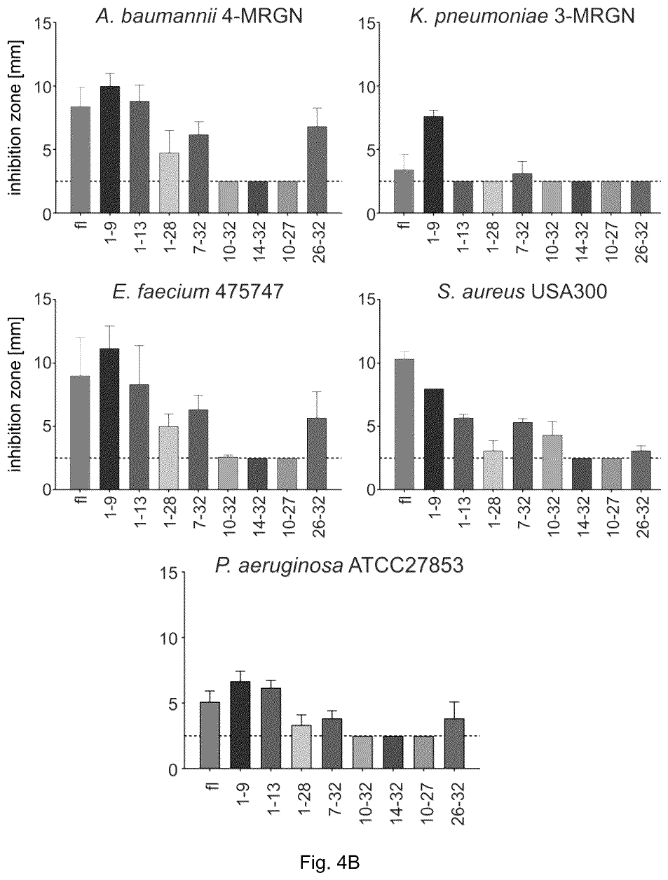

Next, the antimicrobial activity against pathogenic Gram-positive and Gram-negative bacteria (FIGS. 4A and B) was investigated. Here it was found, similar to the previous tested commensal bacteria, that the fragments were antimicrobially active but that the anti-microbial effect varied dramatically among the fragments. Surprisingly it was observed that HD-5₁₋₉, HD-5₁₋₁₃, HD-5₇₋₃₂ and HD-5_{fl} had a strong effect on the growth of every tested bacterium, while other fragments like HD-5₁₋₂₈, HD-5₁₀₋₃₂ and HD-5₂₆₋₃₂ were only minimally active or more selective in terms of different strains. In contrast, HD-5₁₄₋₃₂ and HD-5₁₀₋₂₇ were inactive against the tested bacteria under tested conditions. Of interest, the activity of HD-5₁₀₋₃₂ was limited to the two Gram-positive bacteria. Consistent with the findings described in the first experiment (FIG. 3), and in terms of their efficiency to kill commensals, HD-5₁₋₉ was active against all tested strains independent of Gram status, suggesting that this specific fragment is highly protective against bacterial barrier breach. In contrast, HD-5₁₋₁₃ potently regulated tested pathogenic bacteria, with the exception of K. pneumoniae 3-MRGN. Altogether these results indicate that a proteolytic digestion of Paneth-cell HD-5 but not HD-6 leads to the generation of a number of short and active antimicrobial fragments. The significance of this surprising finding may prove highly important because these fragments broaden the antimicrobial variance based on single full length peptide depending on local environmental conditions.

HD-5 Fragments and their Minimal Inhibitory Concentration Against Antibiotic-Resistant Bacteria

In order to further investigate the potential of these fragments for a potential antibiotic therapeutic use, we measured the minimal inhibitory concentration (MIC) of our peptides against different antibiotic-resistant Gram-negative and Gram-positive bacterial strains. The results of the previous described RDA experiment indicated a promising antimicrobial activity against commensal and pathogenic bacteria. To gain a more detailed understanding of the antimicrobial abilities of the different HD-5 fragments, we performed turbidity broth assays to determine the MIC of these peptides. We determined the MIC as the concentration in which during all experiments no bacterial growth was detectable after 12 hours. Using this parameter we were able to detect antimicrobial activity of HD-5₁₋₉, HD-5₁₋₁₃, HD-5₁₋₂₈, HD-5₇₋₃₂ and HD-5₁₀₋₃₂ (see Table 2 below).

TABLE 2
MIC of HD-5 fragments in μM and μg/ml against pathogenic bacteria. Each experiment
was carried out at least three times. The MIC was determined as the concentration
without any bacterial growth in every experiment after 12 hours of incubation.

| | Bacteria | | | | | | | | | |
|---|---|---|---|---|---|---|---|---|---|---|
| | A. baumannii | | K. pneumoniae | | P. aeruginosa | | E. faecium | | S. aureus | |
| | 4-MRGN | | 3-MRGN | | ATCC 27853 | | 475747 | | USA300 | |
| | MIC | MIC | MIC | MIC | MIC | MIC | MIC | MIC | | MIC, |
| Peptides | μM | μg/ml | μM | μg/ml | μM | μg/ml | μM | μg/ml | μM | μg/ml |
| fl | 6.25 | 22.4 | — | — | 25 | 89.7 | 3,125 | 11.2 | 3,125 | 11.2 |
| 1-9 | 25 | 25.75 | — | — | 50 | 51.5 | 12.5 | 25.75 | 50 | 51.5 |
| 1-13 | >100 | >146 | — | — | >100 | >146 | — | — | — | — |
| 1-28 | >100 | >311 | — | — | >100 | >311 | 100 | 311 | — | — |
| 7-32 | 25 | 72.25 | — | — | 50 | 144.5 | 25 | 72.2 | — | — |
| 10-32 | >100 | >257 | — | — | >100 | >257 | 100 | 257 | — | — |
| 14-32 | — | — | — | — | >100 | >214.4 | — | — | — | — |
| 10-27 | — | — | — | — | — | — | — | — | — | — |
| 26-32 | — | — | — | — | — | — | — | — | — | — |

Comparing the MIC (in μM) of these fragments with the full-length peptide, most of the defensin fragments surprisingly were as active, as the full length peptide. However, when the μg/ml MIC concentrations of the peptide fragments were compared, less was needed and—based on this concentration—especially HD-5₁₋₉ was more active than the full-length peptide. Of note, the observed MIC's were relatively high, except for HD-5₁₋₉ for A. baumannii 4-MRGN and E. faecium 475747, indicating that the role of antimicrobial active substances in the intestinal barrier is restricted to areas with high concentrations like the crypts. To clarify their functional capacity in vivo it was aimed to investigate their intestinal microbiome modulatory functions, in particular because Paneth cells are naturally located in the intestinal tract.

The antimicrobial activity was determined by the minimal inhibition concentration against Gram-negative and -positive bacteria as well as Candida species (Table 3) to investigate if the HD5₁₋₉ Mode of Action was disulfide bond and charge dependent.

Thus, several variants of HD5₁₋₉ were synthesized including an exchange of cysteines with α-aminobutyric acid (Abu) and arginine substitutions with citrulline (Cit). Additionally, a reverse (RGTRCYCTA) and random (CTRATYCRG) amino acid structure of HD5₁₋₉ was tested. HD5₁₋₉ displayed strong bactericidal effects against E. coli BW 25113 whereas HD5₁₋₉ was less antimicrobial active against S. enterica serovar Enteritidis. Similar effects were observed for the reversed variant RGTRCYCTA against both Gram-negative bacteria. The HD5₁₋₉ variants surprisingly lost their antimicrobial activity against tested Gram-negative bacteria if the sequence was random and when lacking either cysteine or arginine amino acids. For both Staphylococcus species no bactericidal effects were determined for HD5₁₋₉ or its variants. In contrast, HD5₁₋₉ displayed a strong bacterial activity against E. faecalis. However, cysteine and arginine substitutions of HD5₁₋₉ lead to a complete dissolving of bactericidal effects as well as random sequences RGTRCYCTA and CTRATYCRG. Unlike E. faecalis, neither HD5₁₋₉ nor its variants showed bactericidal effects against E. faecium. The antimicrobial activity of HD5₁₋₉ and variants was additionally investigated against two Candida species after 24 h. Inhibition of fungal growth could be observed for C. tropicalis displaying a low antimicrobial spectrum. Cysteine and arginine substitutions of HD5₁₋₉ had no effect on fungal growth inhibition. Similar effects were observed for C. albicans while HD5₁₋₉ and variants displayed no antimicrobial activity. In summary, results strongly emphasize the importance of present cysteine and arginine residues and an original amino acid structure of HD5₁₋₉ in order to induce antimicrobial effects.

Table 3 below shows the antimicrobial activity of HD5₁₋₉ against tested bacteria and Candida species. The minimal inhibition concentration (MIC) was determined of E. coli BW 25113, S. enterica serovar Enteritidis, S. aureus SA113, S. epidermidis Evans 1916, E. faecalis ATCC 19433, E. faecium ATCC 19434, C. tropicalis ATCC 4563 and C. albicans ATCC 10231 with different HD5₁₋₉ concentrations due to the optical density after 18 h or 24 h. Results from three independent experiments are presented:

| | Gram negative | | Gram positive | | | | Candida | |
|---|---|---|---|---|---|---|---|---|
| HD5₁₋₉ and | E. | S. | S. | S. | E. | E. | C. | C. |
| variants | coli | enterica | aureus | epidermidis | faecalis | faecium | tropicalis | albicans |
| ATCYCRTGR | XX | X | | | XX | | XX | |
| SEQ ID NO 1 | | | | | | | | |
| AT-Abu-YCRTGR | | | | | | | | |
| SEQ ID NO 91 | | | | | | | | |
| ATCY-Abu-RTGR | | | | | | | | |
| SEQ ID NO 92 | | | | | | | | |
| AT-Abu-Y-Abu- | | | | | | | | |
| RTGR | | | | | | | | |
| SEQ ID NO 93 | | | | | | | | |
| ATCYC-Cit-TGR | | | | | | | | |
| SEQ ID NO 94 | | | | | | | | |
| ATCYCRTG-Cit | | | | | | | | |
| SEQ ID NO 95 | | | | | | | | |
| ATCYC-Cit-TG-Cit | | | | | | | | |
| SEQ ID NO 96 | | | | | | | | |
| RGTRCYCTA | XX | X | | | | | | |
| SEQ ID NO 97 | | | | | | | | |
| CTRATYCRG | | | | | | | | |
| SEQ ID NO 98 | | | | | | | | |

XX High activity
X Low activity
No activity

Table 4 below shows also the effect of HNP-4 fragments according to the invention. From this table can be seen that the HNP-4 peptide fragment according to the invention displays similar anti-microbial behavior as the HD-51-9 peptide.

TABLE 4
Table 4 below shows the antimicrobial activity of HD5₁₋₉,
HD5_{1-9;mod}, HNP-4₁₋₁₁ and HNP-4_{1-11;mod}, against tested bacteria and
Candida albicans. The minimal inhibition concentration (MIC)
was determined of A. baumannii 4-MRGN, A. baumannii DSM
30007, E. faecium 475747, E. faecium DSM 20477, K. pneumoniae
3-MRGN, K. pneumoniae DSM 301404, P. aeruginosa 4-MRGN,
P. aeruginosa ATCC 27853, P. aeruginosa PAO1, P. aeruginosa XPAT1,
P. aeruginosa XPAT2, S. aureus USA300, S. aureus ATCC 25923,
S. entertidis, E. coli BW 25113, Y. enterocolitica, and C. albicans
525L with different petide concentrations due to the optical density
after 12 h. Results from three independent experiments are presented:

| MIC after 12 hours in μM | HD-5₁₋₉ | HD-5_{1-9mod} |
|---|---|---|
| A. baumannii 4-MRGN | 25 | 12.5 |
| A. baumannii DSM 30007 | 100 | 25 |
| E. faecium 475747 | 12.5 | 3.125 |
| E. faecium DSM 20477 | 25 | 6.25 |
| K. pneumoniae 3-MRGN | >>> | >>> |
| K. pneumoniae DSM 30104 | >>> | >>> |
| P. aeruginosa 4-MRGN | 100 | 50 |
| P. aeruginosa ATCC 27853 | 50 | 12.5 |
| P. aeruginosa PAO1 | 50 | 50 |
| P. aeruginosa XPAT1 | >100 | >100 |
| P. aeruginosa XPAT2 | >100 | 100 |
| S. aureus USA300 | 50 | 12.5 |
| S. aureus ATCC 25923 | 50 | 12.5 |
| S. entertidis | 50 | >>> |
| E. coli | 25 | 25 |
| Y. enterocolitica | 50-100 | 50-100 |
| C. albicans 529L | >>> | >100 |

### HD-5₁₋₉ Treatment Affects Certain Bacterial Genera, but not the Microbial Diversity

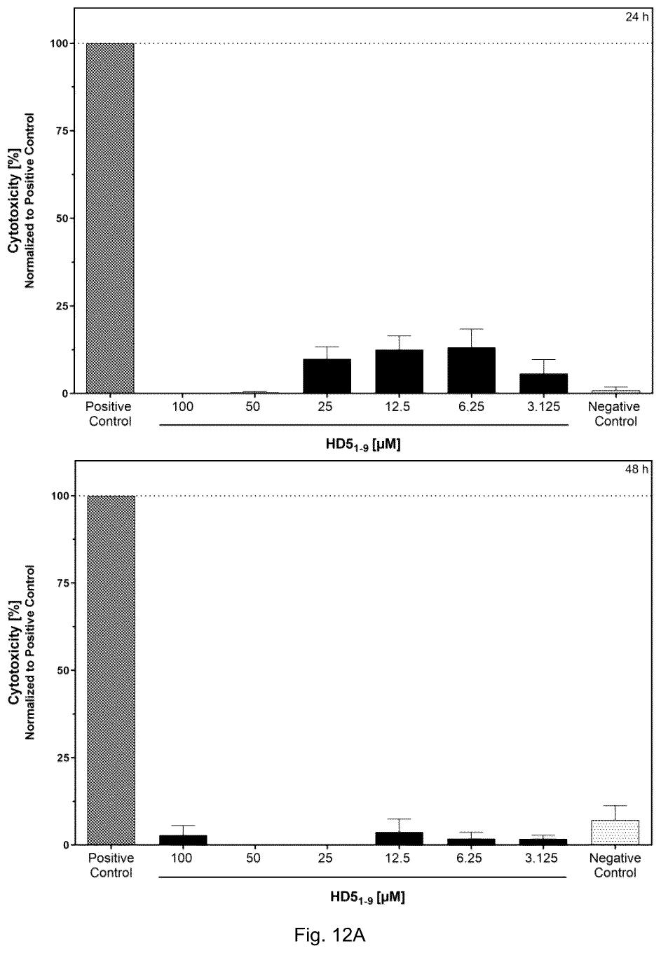

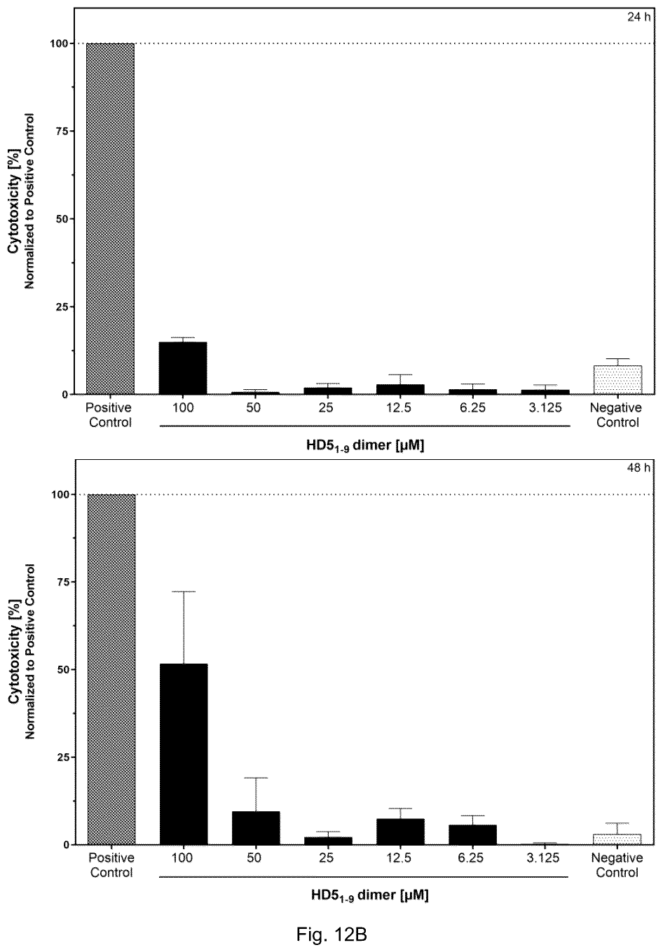

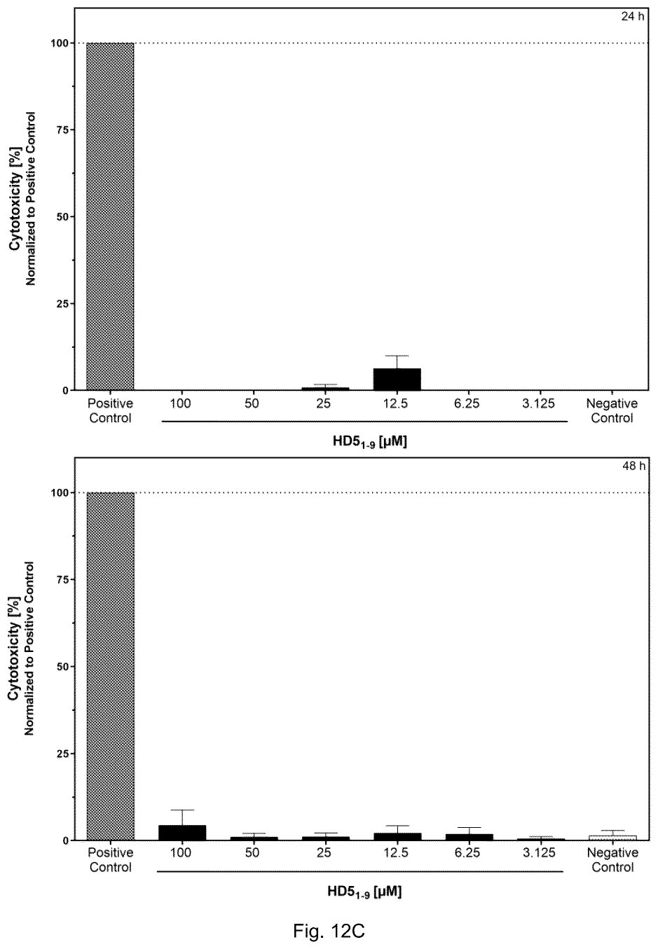

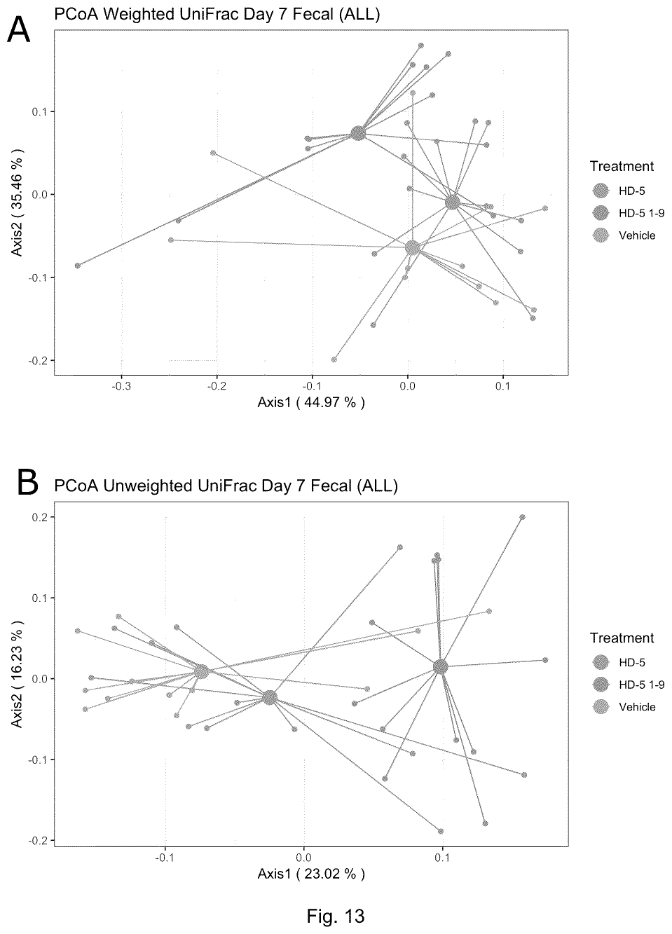

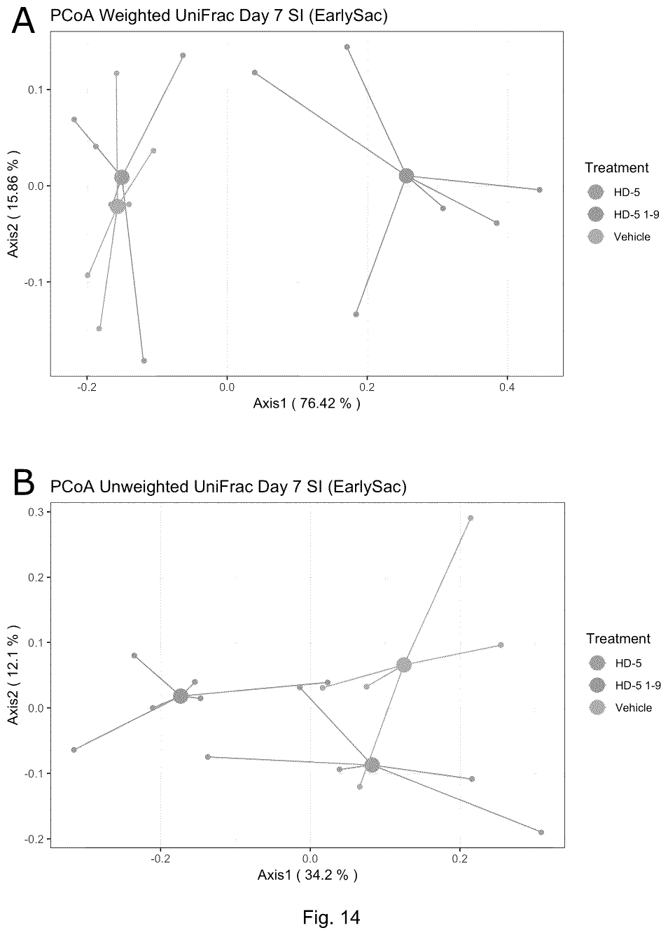

To investigate the microbiome modulatory function of the identified HD-5 fragments, chow fed mice were treated with HD-5₁₋₉ or PBS orally for 7 days (7.19 μg/mouse), followed by 7 days washout. The microbiota composition of the two groups of mice were indistinguishable at baseline (Adonis PERMANOVA using Bray Curtis-distance, p=0.22). After 7 days of HD-51-9 treatment there was a borderline divergence in the overall microbiota composition in fecal samples compared to the control (Adonis PERMANOVA using Bray Curtis-distance, p=0.08) but not compared to the group's baseline microbiota (Adonis PERMANOVA using Bray Curtis-distance, p=0.38, data not shown). As HD-5 is naturally secreted by Paneth cells of the small intestine we therefore investigated the microbial community of the small intestine at necropsy. HD-5₁₋₉ treated mice exhibited no statistical change in the microbial community structure in the small intestine compared to the control after 7 days of washout (Adonis PERMANOVA using Bray Curtis-distance, p=0.09, data not shown). The results were consistent with an initial experiment implying that HD-5₁₋₉ surprisingly does not change the overall fecal microbiota composition in healthy, chow fed mice despite its pronounced anti-microbial efficacy (FIG. 12).

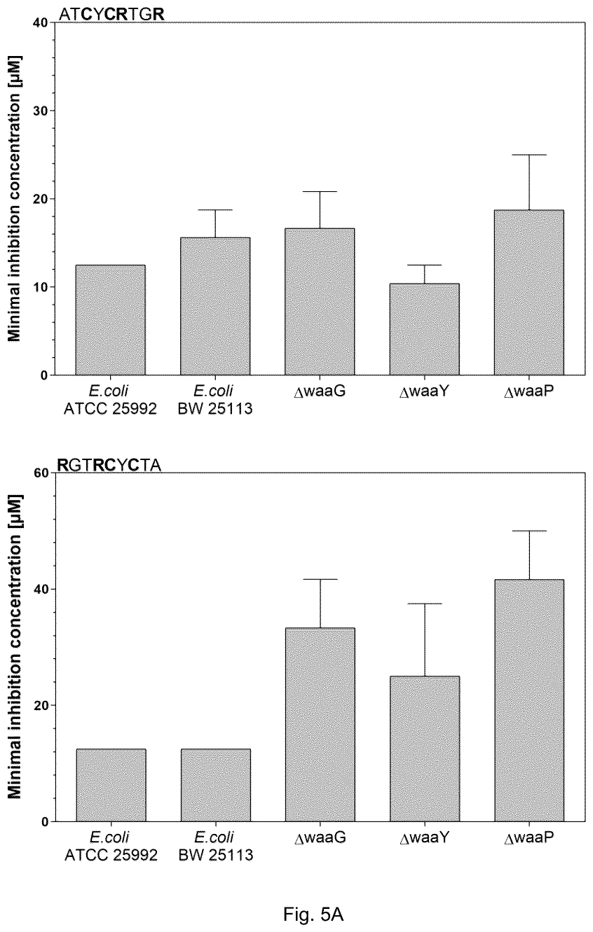

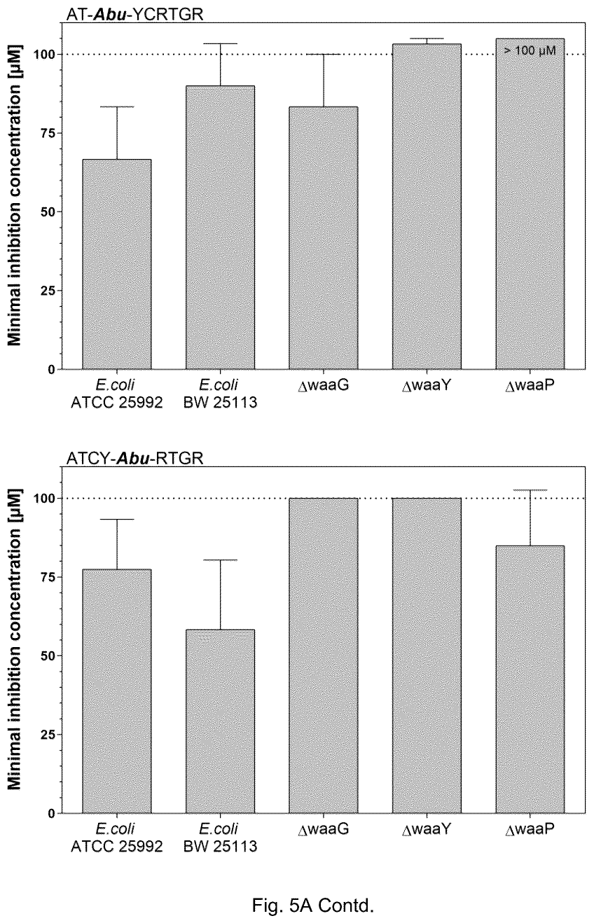

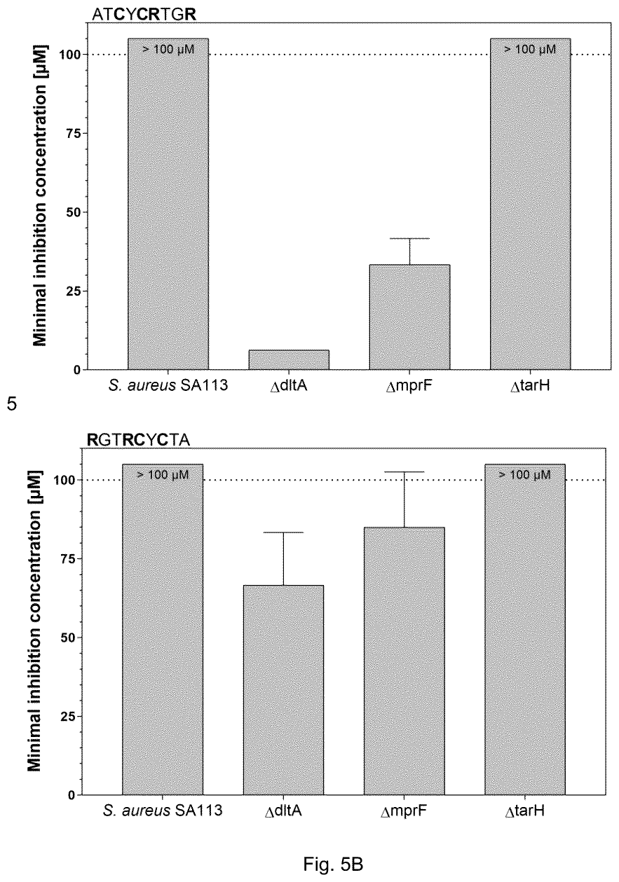

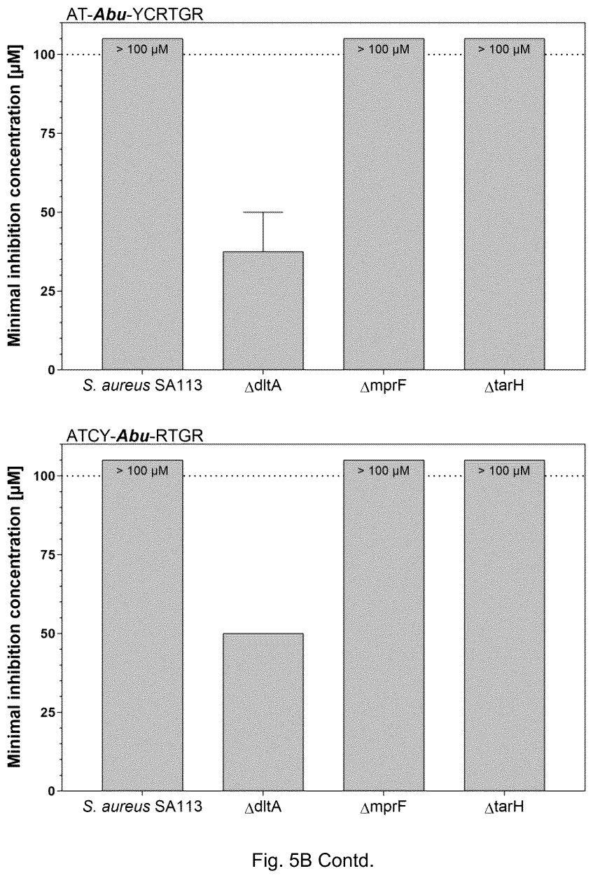

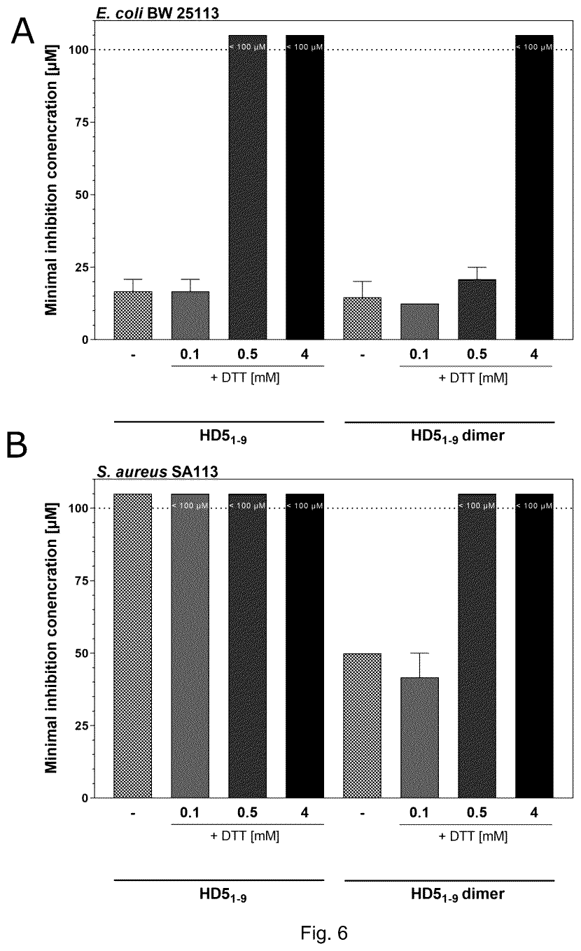

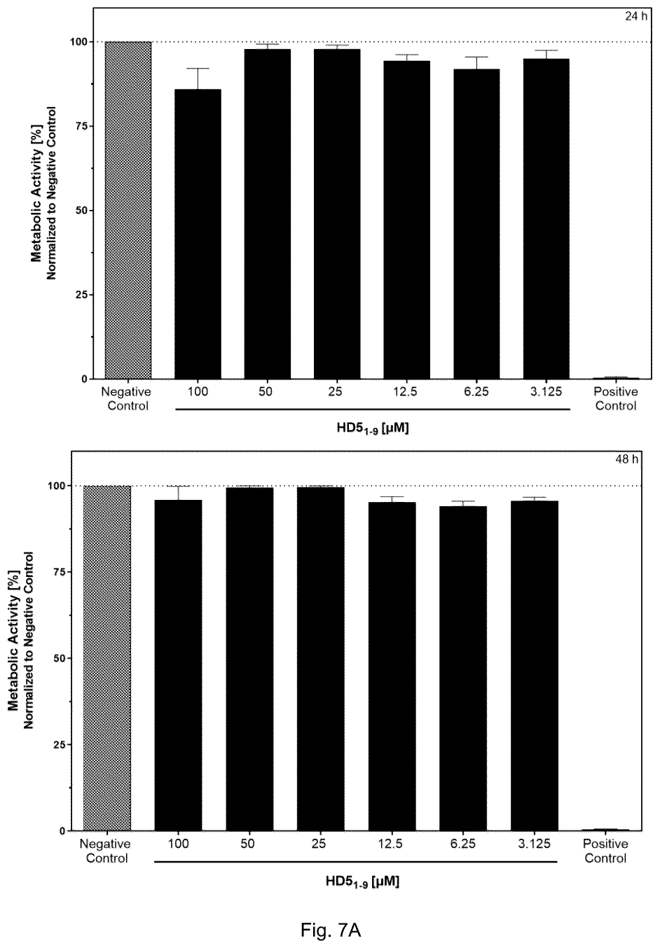

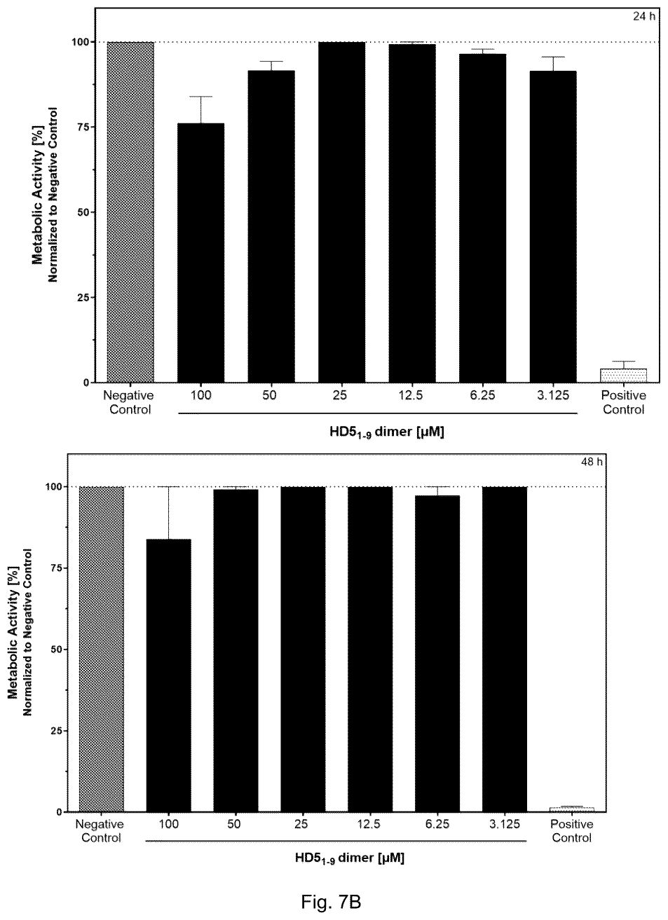

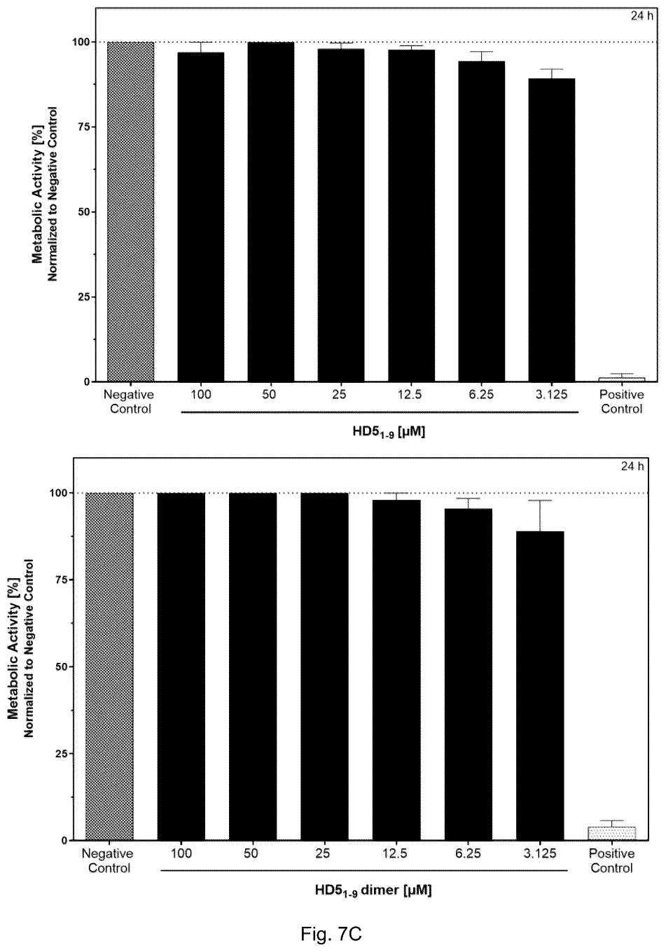

The Shannon diversity of the fecal microbiota composition was equal between the groups at baseline (Wilcoxon test, p=1), and remained similar after 7 days of HD-5₁₋₉ treatment (Wilcoxon test, p=0.18) and after the washout at day 14 (Wilcoxon test, p=0.07) (data not shown). The diversity of the small intestine microbiota was likewise similar between the two groups at day 14 (Wilcoxon test, p=0.45, FIG. 5B right), but not at day 7, where HD-51-9 treated mice surprisingly exhibited increased bacterial diversity compared to vehicle gavaged control mice (p=0.004, data now shown).

The experimental design further allowed to perform paired analyses of the fecal samples using a linear mixed model stratifying for potential co-caging effects and repeated sampling from the same mice. A significant effect of HD-5₁₋₉ on a few low-abundant microbial genera in the fecal samples was identified. More specifically, an increase in relative abundance of genera Parasutterella and Candidatus Stoquefichus was observed while GCA-900066575 of the Lacnospiraceae family and Hydrogenoanaerobacterium were decreased by HD-5₁₋₉ treatment. The genus Akkermansia was borderline increased by HD-5₁₋₉ in the fecal samples (Linear mixed model, p=0.0754, (data not shown), which confirmed the findings of an initial experiment in which Akkermansia was specifically increased by HD-5₁₋₉ treatment (Linear mixed model, p=0.0748). The small intestine microbiota was broadly similar between the two groups at day 14, although the relative abundance of Akkermansia was increased by HD-5₁₋₉ (Linear mixed model, p=0.0085), matching our result from the previous experiment (Linear mixed model, p=0.017. In addition the genus Ruminococcus_1 of the Ruminococcaceae family was increased in relative abundance while Intestinimonas, ASF356 of the Clostridiaceae family, and Ruminococcaceae_UCG_013 of the Ruminococcaceae family were decreased in relative abundances (data not shown).

Combined, these results demonstrate that HD-5₁₋₉ alters the amount of certain low-abundant bacteria in the fecal microbiota, surprisingly without affecting the overall community structure or diversity in healthy chow-fed mice.

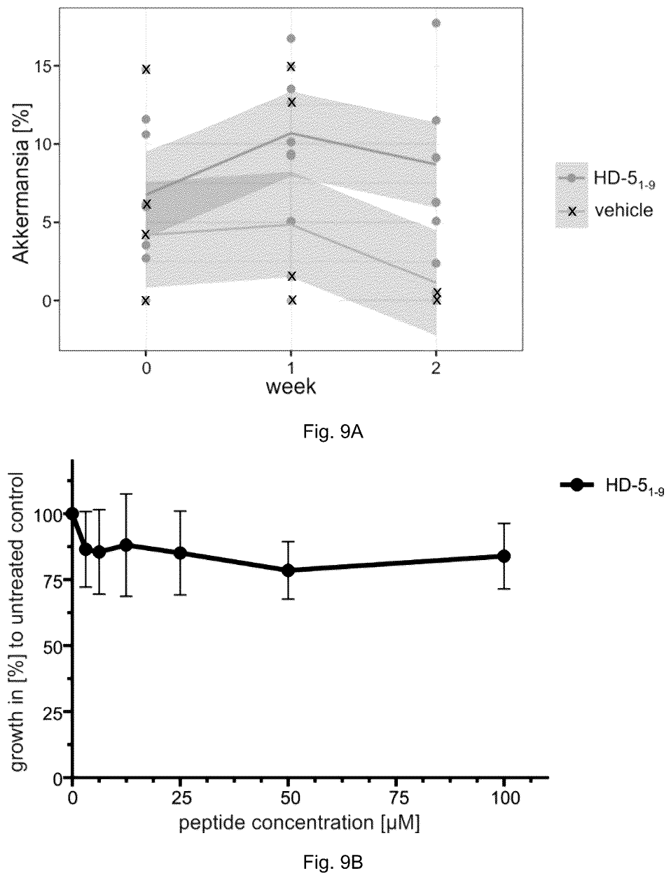

Additionally, it was tested if A. muciniphila is susceptible to HD-5₁₋₉ in a turbidity broth assay. The findings indicate, that even small concentrations of HD-5₁₋₉ decrease the growth of A. muciniphila slightly, but did not kill the bacteria (FIG. 9B).

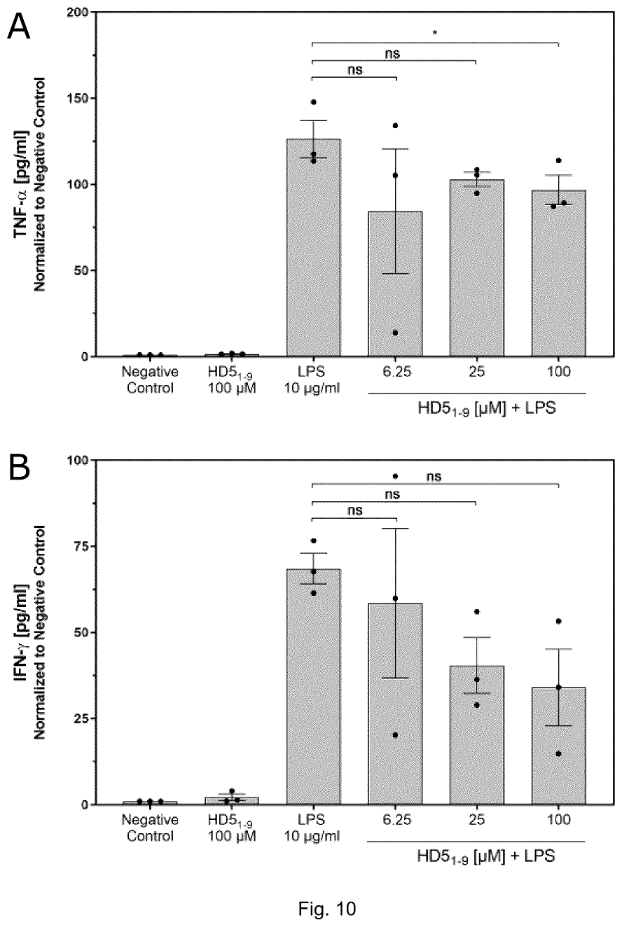

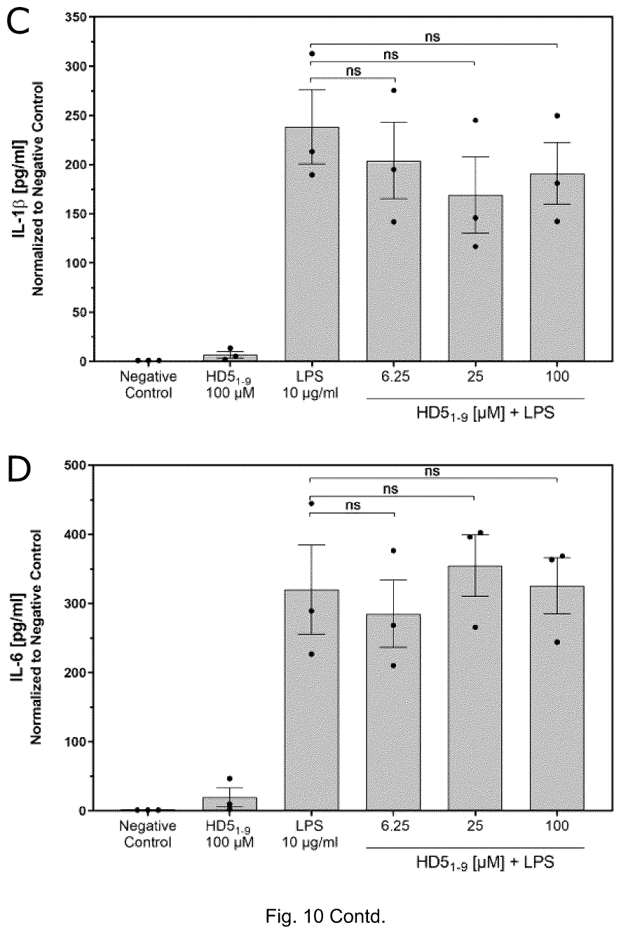

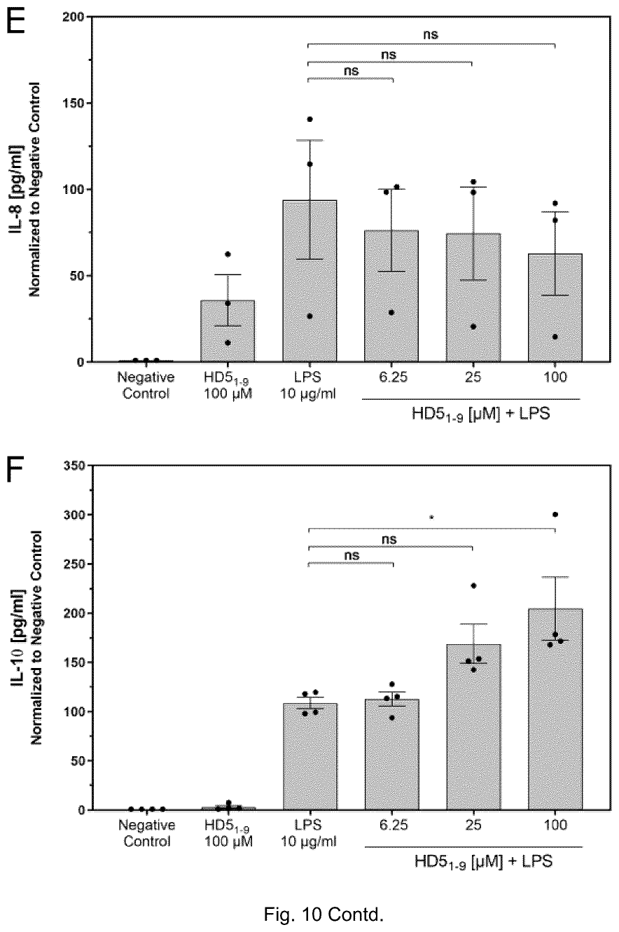

The pro-and anti-inflammatory effect of HD5₁₋₉ was tested and despite the fact that there was a low anti-inflammatory effect with a dose dependent tendency to reduce IFN-γ and IL-8 this was not statistically significant and neither was the dose dependent tendency to increase IL-10 (FIG. 10).

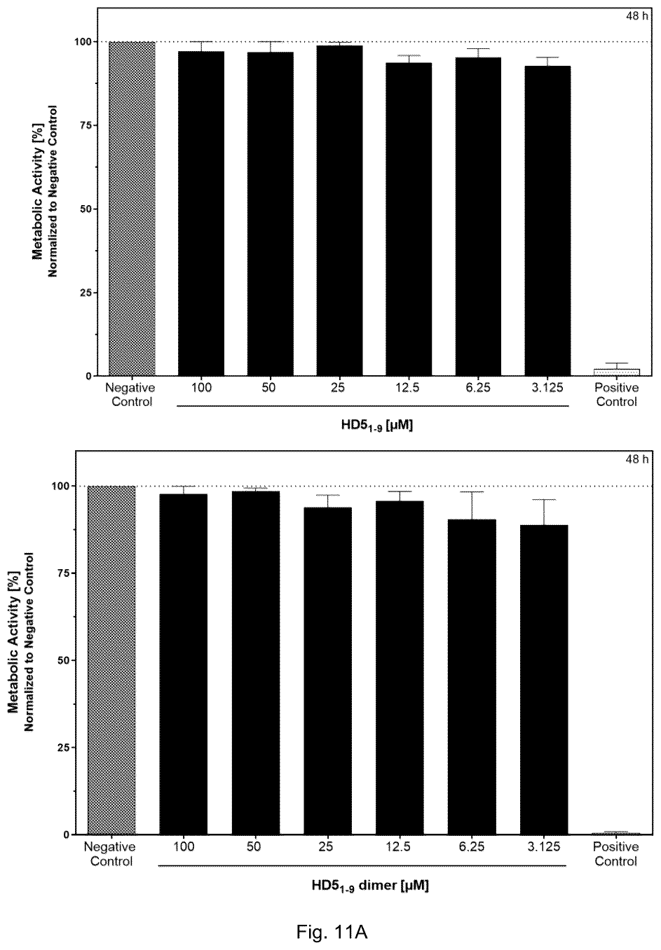

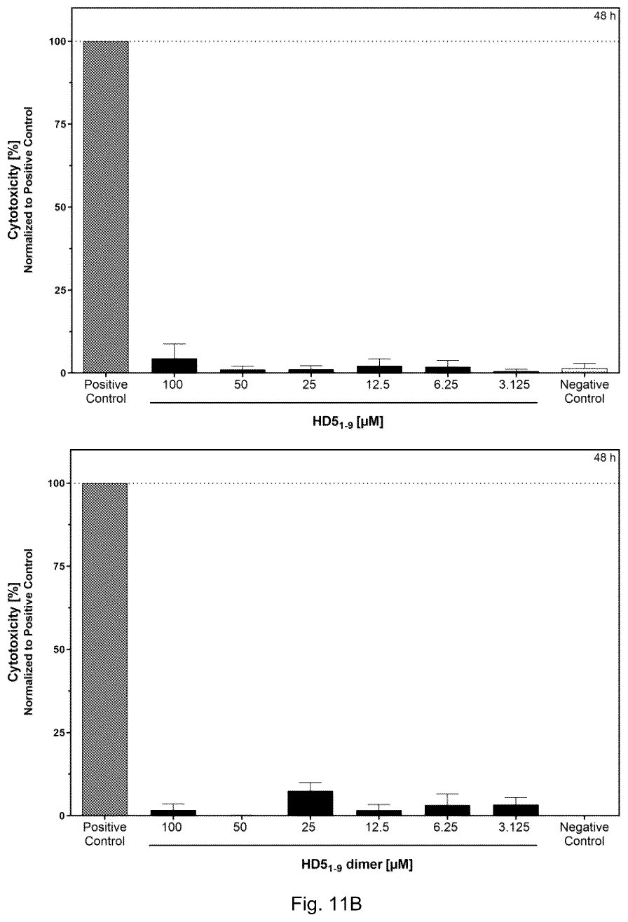

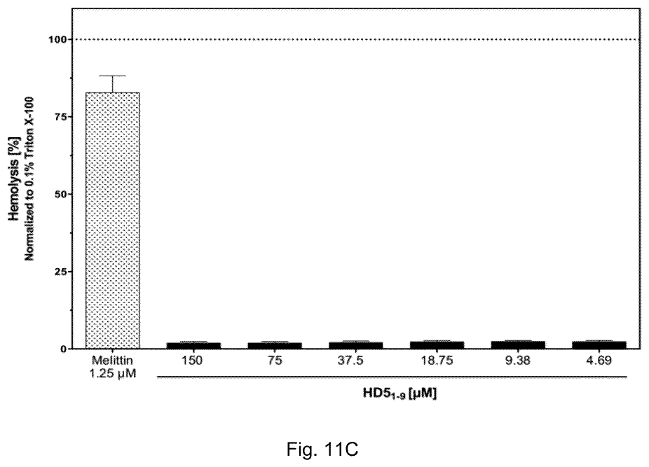

The toxic effect of HD5₁₋₉ was also tested in a number of in vitro experiments (FIGS. 11 and 12) but despite HD5₁₋₉ being a short linear peptide surprisingly no toxicity was observed.

Identification of Cell Wall Targets in Mutants of E. coli BW 25113 and S. aureus SA113

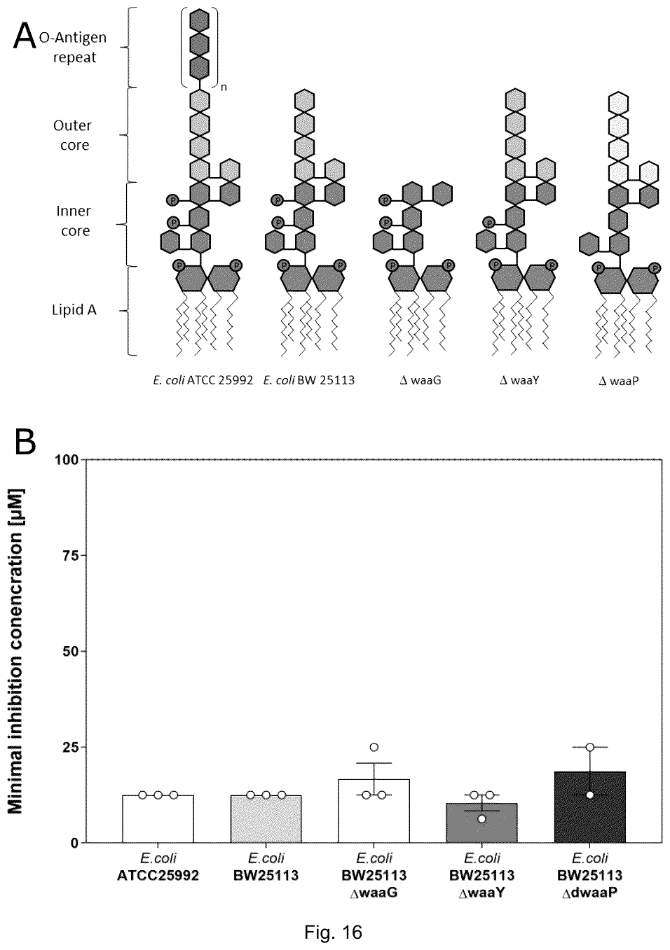

The mode of action of HD5₁₋₉, was identified employing cell wall mutants of E. coli BW 25113 and S. aureus SA113. The aim was to analyze which components of the cell wall are important for binding of HD5₁₋₉ and whether the charge plays a crucial role. A turbidity assay was performed as described above. E. coli strains with different LPS structures were investigated by determining the MIC (FIG. 16). The wild type E. coli BW 25113 as well as E. coli ATCC 25922 were used as controls. The last one contains a similar cell wall composition as E. coli BW 25113 but additionally an O-antigen thus possesses the full-length LPS. The mutant E. coli BW 25113 ΔwaaG contains the same amount of phosphate residues in the inner membrane as the wild type thus possess a similar charge, but the outer core is missing. The mutant ΔwaaY includes an outer core while some phosphate residues in the inner core are missing. The last E. coli mutant ΔwaaP possess an outer membrane as well, but no phosphate residues in the inner membrane leading to a more positive charge of the inner membrane¹⁹.

The antimicrobial activity of HD5₁₋₉ was analyzed against different E. coli strains and mutants. E. coli ATCC 25922 with full-length LPS was as highly susceptible towards HD5₁₋₉ as the tested E. coli BW25113 lacking the O antigen. In order to clarify the function of the outer core during HD51-9 binding to the bacterial cell wall the mutant ΔwaaG was used. Determination of the MIC shows similar antimicrobial effects of HD5₁₋₉ against ΔwaaG like E. coli ATCC 25922 and E. coli BW25113. Surprisingly, E. coli ΔwaaY, lacking one inner core phosphate, and E. coli ΔwaaP, lacking two inner core phosphates, were not more resistant towards HD5₁₋₉. This observation is surprising as it is known that cationic antimicrobial peptides interact with anionic phospholipids in the cell wall due to electrostatic interactions. Lacking this negative charge in the inner core of the cell wall would normally reduce the capability of the peptide to bind on the bacterial cell wall. But HD5₁₋₉ surprisingly shows bactericidal effects at a concentration of approximately 12.5 μM for all the described E. coli mutants. An explanation could be that the antimicrobial activity not only depends on the negative charge of the cell wall but that also an additional binding site must be present in Gram-negative bacteria.

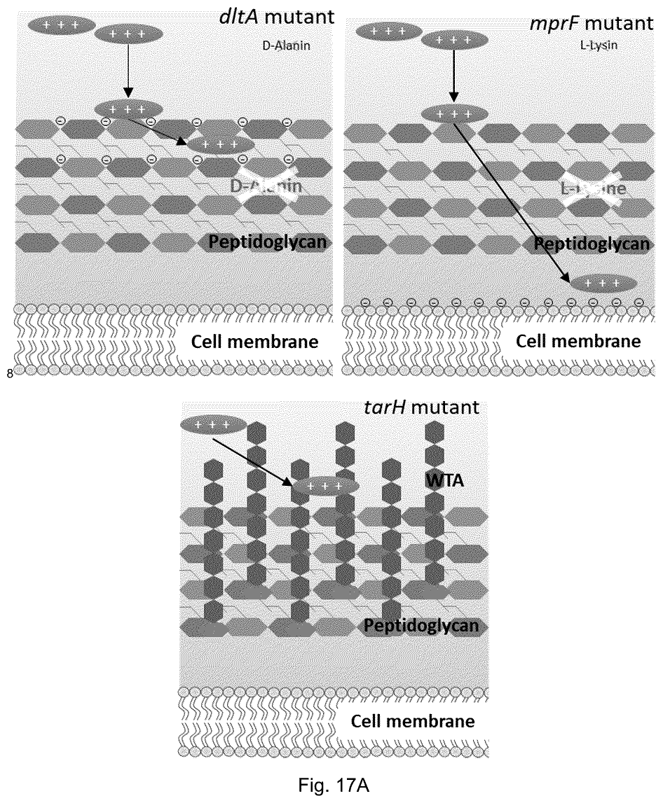

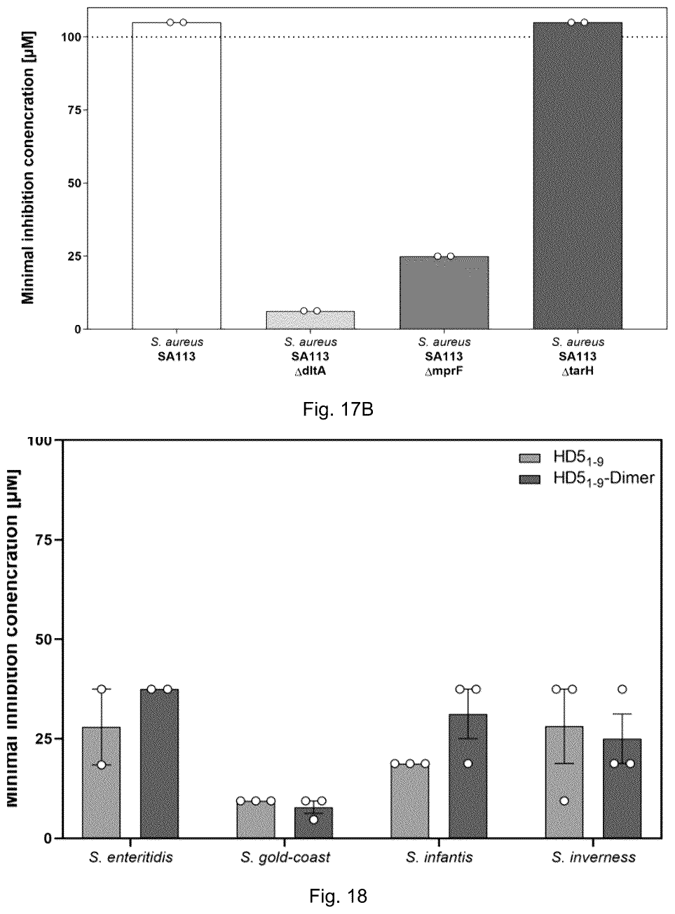

The antimicrobial activity of HD5₁₋₉ and variants was investigated against various S. aureus SA113 strains with different cell wall mutations (FIG. 17). The aim was to investigate how HD5₁₋₉ is capable to bind to the cell wall of Gram-positive bacteria and whether the charge plays a decisive role for the antimicrobial activity of HD5₁₋₉.

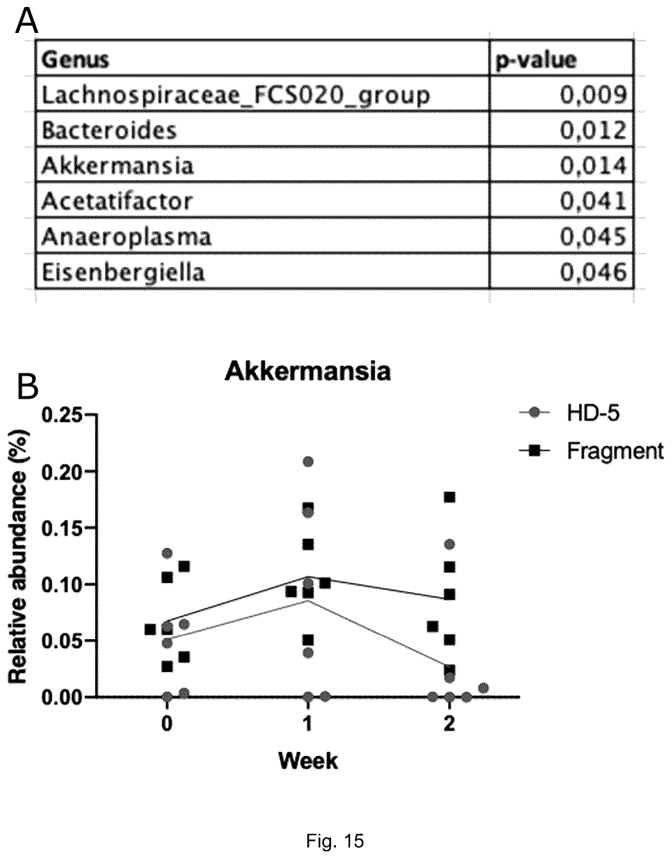

The first mutant ΔditA does not contain D-alanine in the peptidoglycan layer leading to a more negative charge of the peptidoglycan layer²⁰. The mutant ΔmprF lacks L-lysine resulting in a more negative charge of the cell membrane²¹. The last mutant ΔtarH contains additionally wall teichoic acid causing a strengthening of the peptidoglycan²². HD5₁₋₉ showed no growth inhibition against the wild type strain S. aureus SA113 whereas the S. aureus ΔditA mutant containing a more negative peptidoglycan layer was much more susceptible towards HD5₁₋₉ showing a MIC at 6.25 μM (FIG. 15). HD5₁₋₉ also displayed bactericidal effects against the ΔmprF mutant lacking L-lysin, which results in a more negative charge of the bacterial cell membrane. However, surprisingly strengthening of the peptidoglycan layer due to additional teichoic acid in the S. aureus ΔtarH mutant decreased the antimicrobial effects of HD5₁₋₉. These results emphasize the importance of the cell wall charge for the binding of HD5₁₋₉ to Gram-positive bacteria, whereas the cell wall charge seems of less importance for the binding to Gram-negative bacteria.

Characterization of the Antimicrobial Activity of HD5₁₋₉ and HD5₁₋₉-Dimer Against Different Bacteria

HD5 possess leucine residues enabling it to form dimers. Leucine substitutions of HD5 cause a decline of the antimicrobial activity and ability to kill microorganisms indicating that dimerization of HD5 is important for its function (Rajabi et al., 2008, 2012; Szyk et al., 2006)²³⁻²⁵. In addition, cysteine residues of HD5 are capable of composing dimers due to disulfide or hydrogen bonds. Studies showed that cysteine mutations of HD5 influence the oxidative folding, antibacterial activity, Gram-negative bacterial membrane permeabilization as well as proteolytic stability (Wanniarachchi et al., 2011)²⁶.

It is thus reasonable to assume that also HD5₁₋₉ is able to form dimers due to the existence of cysteine residues. Two monomers of HD5₁₋₉ were linked via disulfide bonds resulting in dimerization. The aim was the investigation of the antimicrobial activity of HD5₁₋₉ structured in its dimer form compared to the monomer form against selected bacteria. HLPC and Mass Spectometry identified the dimer as having a MW of 2058 consistent with the existence of one disulphide bridge between the two monomers.

The minimal inhibition concentration was determined in a turbidity assay for Gram-positive bacteria (S. aureus species) and Gram-negative bacteria (Salmonella species) treated with the same concentrations as for HD5₁₋₉.

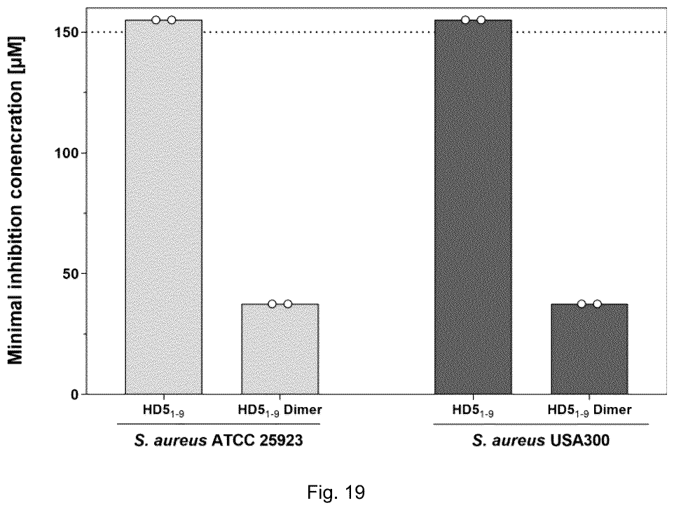

The conducted experiments demonstrated, that the antimicrobial activity of HD5₁₋₉ surprisingly is similar to its dimer form or even better against tested bacteria. The antimicrobial activity of HD5₁₋₉ as well as its dimer form is almost the same against different Salmonella species (FIG. 18). However, surprisingly dimerized HD5₁₋₉ displayed a much better bactericidal activity against S. aureus species compared to the monomer form of HD5₁₋₉ (FIG. 19). This implicates a surprising different mode of action for the two forms of HD5₁₋₉.

Identification of a Novel HNP-4 Fragment after a Tryptic Digestion

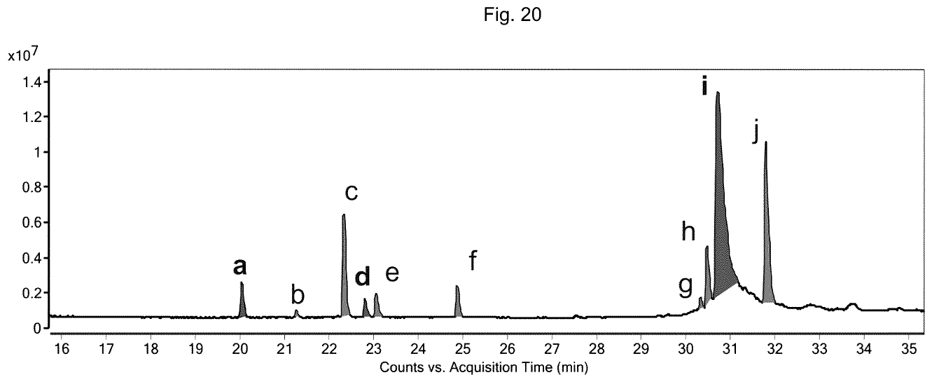

We incubated HNP-4 with 2 mM TCEP to open the disulphide bonds leading to a more linear structure susceptible to proteolytic digest. We analyzed the trypsin incubated reduced HNP-4 via LC/MS methods and were able to detect several fragments (FIG. 20A). According to the observed ions and their mass to charge ratio we were able to clearly identify these fragments, which are mostly located in the N-terminal region based on the cleaving sites of trypsin. As it is commonly accepted that the net charge of AMPs could play an important role to their antimicrobial activity, we focused on HNP-4₁₋₁₁ with a positive net charge of +3.

### Antimicrobial Efficacy of HNP-4₁₋₁₁

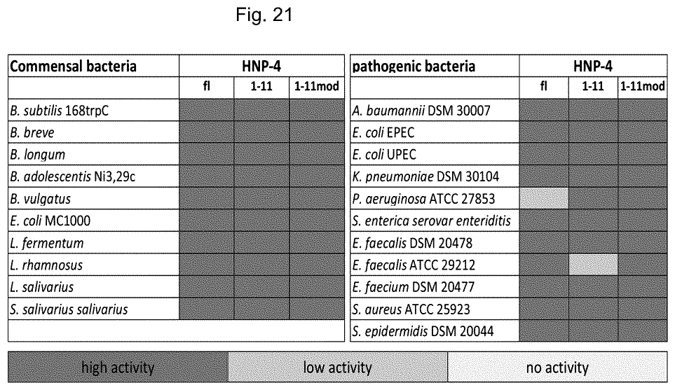

Because the natural stability of short linear peptides is weak, we used an additional modified form of HNP-4₁₋₁₁ (HNP-4_{1-11mod}). Here we exchanged the L-amino acids with D-amino acids and modified the N-terminus (acetylation) and C-terminus (amidation). Both modifications should result in a gain of stability^{27,28}, hence potentially leading to a stronger antimicrobial activity. To analyze the antimicrobial activity of HNP-4_{fl}, HNP-4₁₋₁₁ and HNP-4_{1-11mod} we used RDAs against a subset of different commensal and pathogenic bacteria. All of our tested peptides showed a strong antimicrobial activity against most of the tested bacteria (FIG. 21). While the RDA is the suitable assay to determine a general antimicrobial activity of different peptides, a comparison between different peptides is not possible according to their different abilities (e.g. diffusion) in an agarose gel. We therefore next used a turbidity broth assay to determine the MIC of HNP-4_{fl}, HNP-4₁₋₁₁ and HNP-4_{1-11mod} against pathogenic (some multi-drug resistant) Gram negative and positive bacteria and one fungal strain (Table 5). While all peptides displayed antimicrobial activity against tested bacteria (sole exception: HNP-4_{fl} against K. pneumoniae DSM30104), HNP-4₁₋₁₁ was surprisingly equimolar to HNP-4_{fl}, indicating that the antimicrobial efficacy of the natural complex-to-produce HNP-4_{fl} is solely dependent on the first 11 amino acids (HNP-4₁₋₁₁). Pointing further towards enhanced bactericidal efficacy of this linear fragment, HNP-4_{1-11mod}, which is expected to exhibit increased stability over the non-modified version, was superior to both HNP-4_{fl} and HNP-4₁₋₁₁ with a MIC several fold lower than the one observed for the natural occurring full length peptide. We have thus unleashed the antimicrobial activity of the full length peptide by tryptic digestion whereby we identified a single fragment with a remarkable antimicrobial potential, exceeding that of the full length peptide on molar level. Surprisingly, we observed the antimicrobial efficacy of the peptides to be equally efficient between multi-drug-resistant and non-resistant strains. Thus, a proteolytic digestion of AMPs could be used to generate new active sequences which could lead to new strategies to overcome antibiotic-resistant bacteria.

TABLE 5
Comparison of the MIC of HNP-4_{fl}, HNP-4₁₋₁₁ and HNP-4_{1-11mod}

| | | HNP-4_{fl} | | HNP-4₁₋₁₁ | | HNP-4_{1-11mod} | |
|---|---|---|---|---|---|---|---|
| | | μM | μg/ml | μM | μg/ml | μM | μg/ml |
| MIC after 12 hours | | | | | | | |
| gram | A. baumannii 4-MRGN | 12.5 | 46.4 | 25 | 33.5 | 6.25 | 8.6 |
| negative | A. baumannii DSM30007 | 3.125 | 11.6 | 6.25 | 8.4 | 6.25 | 8.6 |
| | E. coli JM83 | 12.5 | 46.4 | 6.25 | 8.4 | 6.25 | 8.6 |
| | K. pneumoniae 3-MRGN | >>> | >>> | 25 | 33.5 | 6.25 | 8.6 |
| | K. pneumoniae DSM30104 | 100 | 371.5 | 25 | 33.5 | 6.25 | 8.6 |
| | P. aeruginosa 4-MRGN | 50 | 185.8 | 50 | 67.1 | 6.25 | 8.6 |
| | P. aeruginosa ATCC27853 | 6.25 | 23.2 | 12.5 | 16.8 | 6.25 | 8.6 |
| | P. aeruginosa PAO1 | 12.5 | 46.4 | 12.5 | 16.8 | 12.5 | 17.3 |
| | P. aeruginosa XPAT1 | 12.5 | 46.4 | 12.5 | 16.8 | 12.5 | 17.3 |
| | P. aeruginosa XPAT2 | 12.5 | 46.4 | 12.5 | 16.8 | 12.5 | 17.3 |
| | Y. enterocolitica | 12.5 | 46.4 | 12.5 | 16.8 | 6.25 | 8.6 |
| gram | S. aureus USA300 | 6.25 | 23.2 | 50 | 67.1 | 12.5 | 17.3 |
| positive | S. aureus ATCC25923 | 6.25 | 23.2 | 12.5 | 16.8 | 3.125 | 4.3 |
| | S. enterica serovar | 100 | 371.5 | 25 | 33.5 | 12.5 | 17.3 |
| | enteriditis | | | | | | |
| | E. faecium 475747 | 3.125 | 11.6 | 3.125 | 4.2 | 3.125 | 4.3 |
| | E. faecium DSM20477 | 6.25 | 23.2 | 12.5 | 16.8 | 6.25 | 8.6 |
| MIC after 24 hours | | | | | | | |
| | C. albicans 529L | 50 | 185.8 | 100 | 134.2 | 25 | 34.6 |

We determine the MIC as the lowest concentration without detectable bacterial growth in all experiment after 12 hours of incubation at 37° C. For C. albicans we used 24 hours of incubation..
Each experiment was carried out at least three independent times.
The concentration is expressed both in molarity (μM) and mass (μg/ml).

Cytotoxic and Hemolytic Effects of HNP-4₁₋₁₁ and HNP-4_{1-11mod}

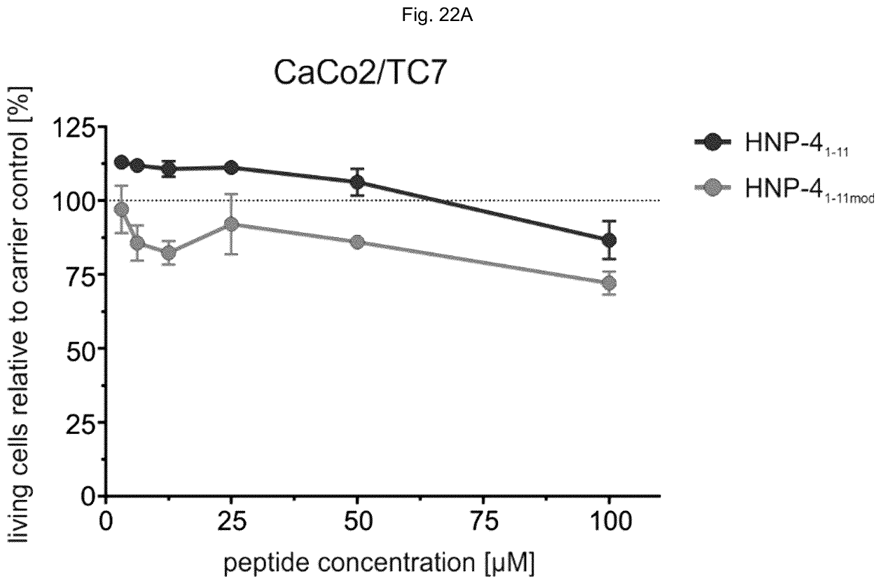

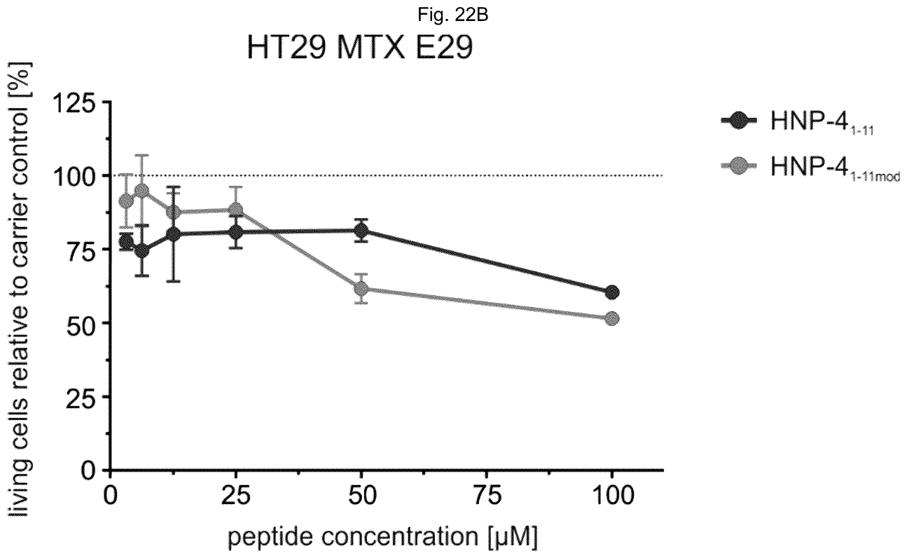

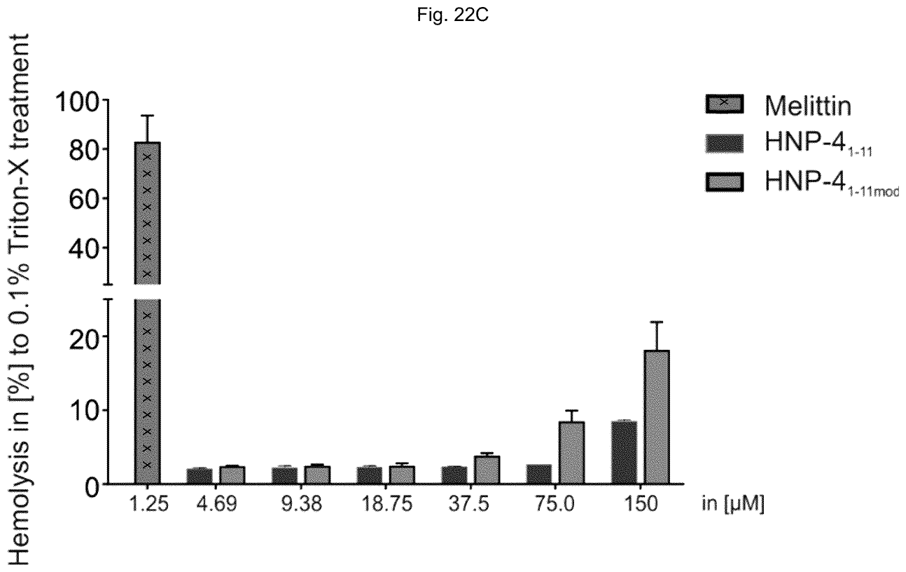

To determine the potential of HNP-4₁₋₁₁ and HNP-4_{1-11mod} for an in vivo application as therapeutic agents, we used two different cell lines to investigate their cytotoxic abilities. While we only observed minor cytotoxic effects on CaCo2/TC7 cells at higher peptide concentration (FIG. 22A), HT29 MTX E29 cells were more susceptible to both 10 tested peptide-derivates (FIG. 22B). Importantly, at lower concentrations (e.g. 12.5 μM, where HNP-4_{1-11mod} has a strong antibacterial effect), the fragments exhibited only modest cytotoxicity. We additionally examined the hemolytic activity of said peptides (FIG. 22C). While HNP-4_{1-11mod} has a 20% hemolytic effect at 150 μM (by far exceeding the highest concentration needed for bactericidal efficacy) there were negligible toxicity at ≤18.75 μM, i.e. the highest biological relevant concentration. Thus, compared to the honey bee toxin, Melittin, which showed an 80% hemolytic effect at 1.25 μM both HNP-4₁₋₁₁ and HNP-4_{1-11mod} appeared with low hemolytic activity. In conclusion, the cytotoxic concentrations identified were magnitudes higher the corresponding bactericidal concentration.

### SUMMARY

Within the study and invention as presented above several new findings regarding alpha-defensins and their susceptibility to proteases were made. Indicating from the in silico digest of HD-6, it was rather surprising that HD-6 was unaffected by the naturally occurring proteases in the duodenal fluid. Unlike HD-6, HD-5 was degraded and its fragments surprisingly contained antimicrobial activity against commensal and pathogenic bacteria.

Determination of the antimicrobial spectrum of the HD-5 fragments revealed a high antimicrobial activity against both commensal and pathogenic bacteria. The antimicrobial spectrum was different from the spectrum of the mother peptide as well as different between the HD5 fragments, and these HD-5 fragments thus seem to add additional bacterial killing or microbiota modulation capability. This interesting and surprising phenomenon also contributes to the understanding of how a few intestinal defensins can modulate or support very different commensal colonizations in different parts of the intestine.

Within the present study and the invention, it was shown that HD5₁₋₉, HD5₁₋₁₃, HD5₁₋₂₈, HD5₇₋₃₂, HD5₁₀₋₃₂, HD5₁₄₋₃₂, HD5₁₀₋₂₇ and HD5₂₆₋₃₂ possessed microbiota modulating effects. Beside the effects on some low abundant bacterial strains, the present results showed that surprisingly only mice treated orally with HD5₁₋₉ had an increased amount of A. muciniphila compared to non-treated mice, moreover different to other bacteria A. muciniphila was not susceptible to HD5₁₋₉ in a turbidity broth assay, fitting to the findings that Akkermansia sp. was increased in the microbiome analysis. Such a surprising influence on A. muciniphila has not previously been described for the full length in the HD-5 peptide. This underlines the different spectra between the full-length peptides and its fragments.

The present study and the invention emphasize the importance of the cell wall charge for the binding of HD5₁₋₉ to Gram-positive bacteria, whereas the cell wall charge seems of less importance for the binding to Gram-negative bacteria. The conducted experiments further surprisingly demonstrated that dimerized HD5₁₋₉ displayed a much better bactericidal activity against S. aureus species compared to the monomer form of HD5₁₋₉. This implicates a different mode of action for the two forms of HD5₁₋₉. HNP-4₁₋₁₁ was surprisingly equimolar to HNP-4_{fl}, indicating that the antimicrobial efficacy of the natural complex-to-produce HNP-4_{fl} is solely dependent on the first 11 amino acids (HNP-4₁₋₁₁). Surprisingly and pointing further towards enhanced bactericidal efficacy of the linear fragment, HNP-4_{1-11mod}, which was expected to exhibit increased stability over the non-modified version, was superior to both HNP-4_{fl} and HNP-4₁₋₁₁ with a MIC several fold lower than the one observed for the natural occurring full length peptide. We have thus unleashed the antimicrobial activity of the full length HNP-4 peptide by tryptic digestion, whereby we have identified a single fragment with a remarkable antimicrobial potential, exceeding that of the full length peptide on molar level. Surprisingly, we observed the antimicrobial efficacy of the peptides to be equally efficient between multi-drug-resistant and non-resistant strains.

Beside their microbiota modulating abilities another important field for antimicrobial active peptides is the rapidly increasing number of antibiotic-resistant bacteria. The antimicrobial spectrum of the peptide fragments identified herein allows to use these peptides as a source of new antibiotics against multi drug resistant bacteria. Also, the discovery of these easy and cheap to produce peptide fragments is a new alternative approach to therapeutically manipulate microbiome composition and treat Paneth cell associated diseases such as Crohn's disease of the small intestine.

### REFERENCES

1. Lehrer, R. I. & Lu, W. a-Defensins in human innate immunity. Immunol. Rev. 245, 84-112 (2012).
2. Ericksen, B., Wu, Z., Lu, W. & Lehrer, R. I. Antibacterial Activity and Specificity of the Six Human α-Defensins. Antimicrob. Agents Chemother. 49, 269-275 (2005).
3. Schroeder, B. O. et al. Reduction of disulphide bonds unmasks potent antimicrobial activity of human β-defensin 1. Nature 469, 419-423 (2011).
4. Chu, H. et al. Human a-defensin 6 promotes mucosal innate immunity through self-assembled peptide nanonets. Science 337, 477-481 (2012).
5. Schroeder, B. O., Stange, E. F. & Wehkamp, J. Waking the wimp: redox-modulation activates human beta-defensin 1. Gut Microbes 2, 262-266 (2011).
6. Wendler, J. et al. Bacterial periplasmic oxido-reductases are essential for the activity of oxidized human antimicrobial β-defensin 1. Infect. Immun. IAI.00875-17 (2018) doi: 10.1128/IAI.00875-17.
7. Stawikowski, M. & Fields, G. B. Introduction to peptide synthesis. Curr Protoc Protein Sci Chapter 18, Unit 18.1 (2012).
8. Davis, L. Basic Methods in Molecular Biology. (Elsevier, 2012).
9. Sambrook, J., Fritsch, E. F. & Maniatis, T. Molecular cloning: a laboratory manual. Molecular cloning: a laboratory manual. (1989).
10. Rowe, R. C., Sheskey, P. J., Cook, W. G. & Fenton, M. E. Handbook of Pharmaceutical Excipients. (Pharmaceutical Press, 2012).
11. Schroeder, B. O. et al. Reduction of disulphide bonds unmasks potent antimicrobial activity of human β-defensin 1. Nature 469, 419-423 (2011).
12. Lehrer, R. I., Rosenman, M., Harwig, S. S., Jackson, R. & Eisenhauer, P. Ultrasensitive assays for endogenous antimicrobial polypeptides. J. Immunol. Methods 137, 167-173 (1991).
13. Oddo, A. & Hansen, P. R. Hemolytic Activity of Antimicrobial Peptides. Methods Mol. Biol. 1548, 427-435 (2017).
14. Callahan, B. J. et al. DADA2: High-resolution sample inference from Illumina amplicon data. Nat. Methods 13, 581-583 (2016).
15. McMurdie, P. J. & Holmes, S. phyloseq: An R Package for Reproducible Interactive Analysis and Graphics of Microbiome Census Data. PLOS One 8, (2013).
16. Paulson, J. N., Stine, O. C., Bravo, H. C. & Pop, M. Differential abundance analysis for microbial marker-gene surveys. Nature Methods 10, 1200-1202 (2013).
17. Oksanen, J. et al. vegan: Community Ecology Package. (2018).
18. ggplot2-Elegant Graphics for Data Analysis (2nd Edition) |Gómez-Rubio| Journal of Statistical Software. doi: 10.18637/jss.v077.b02.
19. Baba, T. et al. Construction of Escherichia coli K-12 in-frame, single-gene knockout mutants: the Keio collection. Mol Syst Biol 2, 2006.0008 (2006).
20. Weidenmaier, C. et al. Lack of wall teichoic acids in Staphylococcus aureus leads to reduced interactions with endothelial cells and to attenuated virulence in a rabbit model of endocarditis. J. Infect. Dis. 191, 1771-1777 (2005).
21. Peschel, A. & Collins, L. V. Staphylococcal resistance to antimicrobial peptides of mammalian and bacterial origin. Peptides 22, 1651-1659 (2001).
22. Wanner, S. et al. Wall teichoic acids mediate increased virulence in Staphylococcus aureus. Nat Microbiol 2, 16257 (2017).
23. Rajabi, M. et al. The conserved salt bridge in human alpha-defensin 5 is required for its precursor processing and proteolytic stability. J. Biol. Chem. 283, 21509-21518 (2008).
24. Rajabi, M. et al. Functional determinants of human enteric a-defensin HD5: crucial role for hydrophobicity at dimer interface. J. Biol. Chem. 287, 21615-21627 (2012).
25. Szyk, A. et al. Crystal structures of human alpha-defensins HNP4, HD5, and HD6. Protein Sci. 15, 2749-2760 (2006).
26. Wanniarachchi, Y. A., Kaczmarek, P., Wan, A. & Nolan, E. M. Human Defensin 5 Disulfide Array Mutants: Disulfide Bond Deletion Attenuates Antibacterial Activity Against Staphylococcus aureus. Biochemistry 50, 8005-8017 (2011).
27. Hong, S. Y., Oh, J. E. & Lee, K. H. Effect of D-amino acid substitution on the stability, the secondary structure, and the activity of membrane-active peptide. Biochem. Pharmacol. 58, 1775-1780 (1999).
28. Brinckerhoff, L. H. et al. Terminal modifications inhibit proteolytic degradation of an immunogenic MART-1 (27-35) peptide: implications for peptide vaccines. Int. J. Cancer 83, 326-334 (1999).

1.-22. (canceled)

23. A peptide consisting of a sequence of 7-9 amino acids, said amino acids comprising the sequence CRTGR (SEQ ID NO: 46), or multimers thereof, or N- or C-terminal non-amino acid modifications thereof.

24. The peptide of claim 23, wherein the peptide consists of 9 amino acids.

25. The peptide of claim 23, wherein the peptide consists of 7 amino acids.

26. The peptide of claim 23, wherein the peptide wherein the peptide consists of or comprises D-and/or L-amino acids.

27. The peptide of claim 23, wherein the peptide comprises N- and/or C-terminal modifications.

28. The peptide of claim 23, wherein the peptide comprises an N-terminal modification selected from acetyl-, formyl-, pyroglutamyl-, fatty acids-, urea-, carbamate-, and alkylamine.

29. The peptide of claim 23, wherein the peptide comprises a C-terminal modification selected from -Amide, -Acid, -N-alkyl-Amide, -Aldehyde, -Ester, -p-Nitroanilide, and -7-Amino-4-Methylcoumarin.

30. The peptide of claim 23, wherein the peptide comprises an N-terminal modification acetyl modification and a C-terminal amide modification.

31. The peptide of claim 23, wherein the peptide is a homodimer linked through a disulfide bond.

32. A method of modulation of the gut microbiome in a subject, said method comprising administration the peptide of claim 23 to said subject.

33. The method of claim 32, wherein the bacterial diversity of the gut microbiome is supported or promoted.

34. The method of claim 32, wherein the bacterial diversity of the small intestine is increased.

35. The method of claim 32, wherein the peptide consists of 9 amino acids.

36. The method of claim 32, wherein the peptide consists of: ATCYCRTGR (SEQ ID No. 1), or Ac-atcycrtGr-NH₂ (SEQ ID No. 5).

37. A method of supporting the bacterial diversity of the gut microbiome in a subject suffering or at-risk of inflammatory bowel disease, coeliac disease, obesity, or type 2 diabetes, said method comprising administration of the peptide of claim 23 to said subject.

38. The method of claim 37, wherein the peptide consists of 9 amino acids.

39. The method of claim 37, wherein the peptide consists of: ATCYCRTGR (SEQ ID No. 1), or Ac-atcycrtGr-NH₂ (SEQ ID No. 5).

40. The method of claim 37, wherein the method is for treatment or prevention of inflammatory bowel disease, coeliac disease, obesity, or type 2 diabetes.
